(19) United States
(12) Patent Application Publication
(10) Pub. No.: US 2014/0212407 A1
(43) Pub. Date: Jul. 31, 2014

(54) TREATMENT OF OBESITY

(71) Applicant: Isis Innovation Limited, Oxford (GB)

(72) Inventors: Amany ELSHORBAGY, Oxfordshire (GB); Helga Margareta REFSUM, Oxfordshire (GB)

(73) Assignee: Isis Innovation Limited, Oxford (GB)

(21) Appl. No.: 14/019,022

(22) Filed: Sep. 5, 2013

Related U.S. Application Data
(63) Continuation of application No. 13/054,891, filed on Jan. 19, 2011, now abandoned.

(30) Foreign Application Priority Data
Jul. 21, 2008 (GB) 0813310.0
Feb. 10, 2009 (GB) 0902157.7

(51) Int. Cl. A61K 38/02; C07K 16/18; G01N 33/52; A61K 31/675; C12N 15/115; A61K 31/197; C12N 15/11; A61K 31/10

(52) U.S. Cl. CPC A61K 38/02; C12N 15/11; C07K 16/18; A61K 31/10; A61K 31/675; C12N 15/115; A61K 31/197; G01N 33/52
USPC 424/130.1; 514/44 R; 514/4.8; 514/711; 514/110; 435/7.91

(57) ABSTRACT

The present disclosure relates to the treatment, e.g. reduction, of body fat mass levels for example in overweight and obese subjects. The present disclosure also relates to weight control in a subject where obesity should be avoided. Disclosed herein are methods of reducing body fat mass e.g. reducing obesity or preventing weight gain and agents used in such methods whereby the agents inhibit a sulphur containing amino acid. Also included in the present disclosure include, without being limited to, methods for determining regimes for the treatment of obesity as well as other subject matter.

### CROSS-REFERENCE TO RELATED APPLICATIONS

This application is a continuation of, and claims priority from, U.S. application Ser. No. 13/054,891, filed Jan. 19, 2011, which is the U.S. National Stage filing under 35 U.S.C. §371 of International Application No. PCT/GB2009/050891, filed Jul. 21, 2009, which claims benefit of the filing date of Great Britain Application No. 0813310.0, filed Jul. 21, 2008, and Great Britain Application No. 0902157.7, filed Feb. 10, 2009.

The present disclosure relates to the treatment, e.g. reduction, of body fat mass levels for example in overweight and obese subjects. The present disclosure also relates to weight control in a subject where obesity should be avoided. Disclosed herein are methods of reducing body fat mass e.g. reducing obesity or preventing weight gain and agents used in such methods. Also included in the present disclosure include, without being limited to, methods for determining regimes for the treatment of obesity as well as other subject matter.

### BACKGROUND

Obesity is a major public health concern and is becoming increasingly prevalent. Obesity has a number of health risks associated with it, for example, an increased risk of cardiovascular disease, e.g. stroke, hypertension, atherosclerotic disease and congestive heart failure. Obesity also results in a higher risk of diabetes mellitus and certain types of cancer (e.g. uterine, breast, colon and prostate). Over 30,000 deaths a year in England alone can be attributed to obesity and its associated health risks. Furthermore, obesity can have a detrimental effect on a person's quality of life through decreased mobility, limited physical endurance and a lack of emotional well-being.

In addition to diet management and exercise regimes which are recommended to reduce obesity, there are also a number of treatments. However, the approved treatments have a number of side-effects, which are often unpleasant and sometimes dangerous.

One drug approved for the treatment of obesity is orlistat. Orlistat (marketed as Xenical®) works by inhibiting pancreatic lipase. As a result, triglycerides from the diet are prevented from being hydrolyzed into absorbable free fatty acids and are excreted undigested. Side effects of orlistat include steatorrhea (oily, loose stools). This occurs because dietary fat is blocked from being absorbed and so the fat is excreted unchanged in the faeces. Other side effects include fecal incontinence, frequent or urgent bowel movements and flatulence.

Sibutramine is also approved for the treatment of obese patients. Sibutramine is a neurotransmitter reuptake inhibitor that reduces the reuptake of serotonin, norepinephrine and dopamine, thereby increasing the levels of these substances in synaptic clefts and helping enhance satiety. Side-effects such as an increase in blood pressure have been reported in a class of patients treated with sibutramine.

Obesity can also been treated surgically. So-called bariatric surgery is primarily used in morbidly obese patients to reduce the amount of food that can be accommodated by the patient's stomach or be absorbed from the gastrointestinal tract. As with any surgery, bariatric surgery involves risk of infection and other complications. These complications often have a higher occurrence in these patients due to the obesity, and therefore often poor health, of the patient. Due to these complications, bariatric surgery is not suitable for all patients, particularly those with heart and lung diseases. A problem after surgery is a wide range of nutritional deficiencies. Furthermore, even after successful weight loss, weight gain is common, and is associated with long term morbidity. Bariatric procedures in current use include gastric bypass, laparoscopic adjustable gastric band, vertical banded gastroplasty, and biliopancreatic diversion and switch.

Therefore, there remains a need for treatments for obesity.

### BRIEF SUMMARY OF THE DISCLOSURE

The inventors believe that certain sulphur-containing amino acids affect body fat mass levels. Thus, in its broadest aspect, the present invention is concerned with the management of body fat mass levels, including for example, the treatment of obesity, using an agent which modulates, e.g. reduces or increases, or changes the action of one or more sulphur-containing amino acids. Such action may be, for example, on adipose tissue.

The level of the sulphur-containing amino acid can be determined by, for example, measuring plasma concentrations of the sulphur-containing amino acid. Thus, in one embodiment, the agent affects (e.g. increases or decreases) the plasma concentration of the sulphur containing amino acid.

One such sulphur-containing amino acid is cysteine. Cysteine may also occur in a dimeric form, where the two cysteine molecules are joined by a disulfide bond. This form is known as cystine. Cysteine can also be bound to other sulphur-containing amino acids, cysteine-mixed disulphides. For simplicity, we use the term cysteine to include both cysteine itself as well as cystine and cysteine-mixed disulphides.

The present invention is also concerned with inducing weight gain in an underweight individual comprising increasing cysteine activity, and optionally the activity of other plasma sulphur containing amino acids, of a subject.

The present invention is also concerned with the control e.g. the suppression, of cysteine and/or its downstream products, so as to control body fat mass in an individual. Thus, the present invention is based, at least in part, on the observation that control of cysteine activity in an individual may control body fat mass levels in a subject. In particular, the present invention is concerned with the reduction of body fat mass, including the reduction of obesity by controlling e.g. reducing, the action of cysteine. The agent may, for example, reduce plasma cysteine levels. The present invention is also concerned with the prevention of weight gain in a subject. In particular, the present invention is concerned with the prevention of weight gain in an individual for whom weight gain would be disadvantageous e.g. instances in which the individual suffers from or has a predisposition for a disease which includes cardiovascular disease, diabetes and cancer.

In one aspect of the present invention there is provided an agent for the modulation of a body fat mass level of a subject, wherein said agent inhibits the activity of a sulphur containing amino acid. In one embodiment, the agent is for the treatment of obesity. In one embodiment, the agent is for the reduction of body fat mass in an overweight subject. In an embodiment, the agent inhibits cysteine activity and optionally reduces the plasma cysteine concentration. The agent may be any agent that is capable of decreasing cysteine bioavailability or action, which may result in a reduction in plasma cysteine concentration in a subject. In one embodiment, the agent inhibits cysteine activity on adipocytes and adipose tissue. As used herein, the term “inhibit” includes total or partial inhibition of activity. In one embodiment, the agent inhibits the activity of the sulphur containing amino acid on a specific tissue or cell type and does not necessarily inhibit the effect of the sulphur containing amino acid on other cell types or tissue. In one embodiment, the agent inhibits the action of the sulphur containing amino acid, e.g. cysteine, on adipocytes. In one embodiment, the agent inhibits the action of the sulphur containing amino acid, e.g. cysteine, by inhibiting cellular uptake of the sulphur-containing amino acid.

Cysteine is a non-essential α-amino acid. A schematic representation of the cysteine generation pathway is shown in FIG. 1 which shows that homocysteine, cystathionine, cysteine, glutathione, cysteinylglycine and taurine are all downstream products of methionine.

As discussed above, one way of determining cysteine levels in a subject is by measuring plasma cysteine levels e.g. plasma total cysteine (tCys). As used herein, the term “plasma cysteine” or “tCys” refers to all natural forms of circulating cysteine, such as cysteine (thiol form), cystine (disulfide form), cysteine-mixed disulphides with other thiol compounds, and protein-bound cysteine not in peptide linkage. The term “plasma cysteine” relates to any cysteine form in plasma that can be detected using standard methods for total cysteine measurements. Such methods are described herein. Furthermore, it will be appreciated that, as used herein, the term “cysteine” refers to all forms of cysteine, such as cysteine (thiol form) and cystine (disulfide form.

The agent may be selected from a small molecule, an aptamer, a peptide, a polypeptide, an antibody and antibody fragments. In one embodiment, the agent is for the treatment of overweight or obesity complicated by one or more disorders. The complication may be selected from one or more of the following: cardiovascular disease, diabetes mellitus, dyslipidemias, metabolic syndrome, musculoskeletal pains, arthritis, hypertension, pulmonary hypertension, atherosclerotic disease, congestive heart failure, cancer, breast cancer, uterine cancer, prostate cancer, sleep apnea syndrome, obesity hypoventilation syndrome lower extremity edema, ventral/umbilical hernia, nonalcoholic steato-hepatitis, cholelithiasis, gastroesophageal reflux disease, stress urinary incontinence, psychosocial impairment or depression and polycystic ovarian syndrome.

The present invention provides an agent for the reduction of the amount of body fat mass in a subject, wherein said agent inhibits cysteine action. In one embodiment, the agent reduces the plasma concentration of cysteine in the subject. The subject may be a Down's syndrome sufferer. Down's syndrome sufferers often have increased cysteine production and obesity.

In one embodiment, the agent as described herein reduces or inhibits the activity of cystathionine beta-synthase enzyme. In one embodiment, the agent reduces or inhibits expression of a cystathionine beta-synthase gene. In one embodiment, the agent reduces or inhibits the activity of cystathionine γ-lyase enzyme. In one embodiment, the agent reduces or inhibits expression of a cystathionine γ-lyase gene.

In one embodiment, the agent is for use in combination with a reduction in nutritional uptake by the subject. The agent may be for use in combination with a diet that is low in cysteine-rich foods. Examples of foods low in cysteine are bananas and casein.

In one aspect of the present invention there is provided use, for the manufacture of a medicament for the modulation of body fat mass, of an agent which inhibits the activity of a sulphur containing amino acid. In one embodiment, the medicament is for the treatment of obesity. In one embodiment, the medicament is for the reduction of body fat mass in an overweight subject. In one embodiment, the agent is as described herein. In one embodiment, the medicament reduces the plasma cysteine (tCys).

The medicament may be for administration via a route selected from one or more of the following: oral, parenteral, transdermal, intradermal and intravenous. The medicament may be for repeated administration, wherein optionally the medicament is for administration at least once a day.

In a further aspect of the present invention, there is provided a method of treating obesity comprising administering a therapeutically effective amount of an agent which inhibits the activity of a sulphur containing amino acid to a subject in need thereof. In one embodiment, the method comprises administrating the agent in an amount sufficient to reduce plasma concentration of the at least one sulphur containing amino acid thereof. In one embodiment, the method comprises administering a therapeutically effective amount of an agent which inhibits cysteine action on adipose tissue to a subject in need thereof. The agent may be as described herein.

In one embodiment, the agent is for the treatment of obesity. The term “obesity” as used herein means accumulation of excess fat on the body. Obese persons are often defined as having a body mass index (BMI) of greater than 30. Subjects having BMI between 25 and 30 are considered overweight and in one embodiment, are treated by the agents disclosed herein. The body mass index (BMI) is calculated by dividing an individual's weight in kilograms by the square of their height in metres. BMI does not distinguish fat mass from lean mass and an obese subject typically has excess adipose tissue.

Thus, in one embodiment of the present invention, the subject has a BMI greater than 30. In other embodiments, the subject may have a BMI lower than 30 and has a disease associated with obesity e.g. high blood pressure, diabetes or cardiopulmonary disorders. In one embodiment, the subject has a BMI of 25 or over, e.g. 26, 27, 28, 29, 30 or greater and has no obesity-related co-morbidity. In another embodiment the subject has a BMI of 25 or over, e.g. 26, 27, 28, 29, 30 or greater and optionally has significant co-morbidity such as diabetes, hypertension and/or hypercholesterolemia. In one embodiment, the patient is morbidly obese and has a BMI of 40 or over.

In one embodiment, the subject is obese and/or suffering from complications associated with obesity. In one embodiment, the subject has a Body Mass Index (BMI) of over 25, and preferably over 30.

In an embodiment, the agent is selected from propargylglycine, a non-steroidal anti-inflammatory drug; sulfasalazine, mesna alone or in combination with ifosamide and a dipeptidase inhibitor, e.g. cilastatin. In one embodiment, the method comprises administering the agent orally, transdermally, intravenously or intradermally.

In an alternative embodiment, the subject is not overweight or obese and the agent is for preventing weight gain. In particular, the agent is for preventing weight gain e.g. an increase in fat mass in a subject for whom an increase in body fat mass is disadvantageous. Such subjects include for example individuals suffering from or who are predisposed to suffering from cardiovascular disease e.g. congestive heart failure, hypertension and atherosclerotic disease, diabetes mellitus and individuals who are predisposed to certain forms of cancer e.g. breast cancer, prostate cancer and the like.

In one aspect, the present invention relates to the management of body fat mass in a subject comprising the use of an agent which inhibits cysteine uptake or action. In one embodiment, the present invention is concerned with the treatment of obesity in a subject comprising the use of an agent which inhibits or reduces the level of plasma cysteine, which is measured as total cysteine (tCys). In one embodiment, the obesity is a result of abnormal levels of primary aminothiols e.g. high cysteine concentrations.

In one aspect of the present invention, there is provided a product comprising an agent which modulates cysteine activity. In one embodiment, the agent reduces cysteine activity e.g. on adipose tissue. In one embodiment, the product is a nutraceutical. In one embodiment, the product is a food product.

Certain embodiments of the present invention are therefore concerned with the treatment of overweight and/or obesity. As used herein, the term “treatment” includes the reduction of obesity in a patient. Reduction of obesity may be considered to include a reduction in the body weight of the patient. In one embodiment, the reduction of body weight is at least 2%, e.g. 3%, 4%, or 5% of the total body weight of the patient. In an embodiment, the weight loss may be greater than 5% e.g. 6%, 7%, 8%, 9%, 10%, 15% or more.

Alternatively or in addition, treatment of obesity can be taken to include a reduction in body fat mass in a subject. In one embodiment, the methods and agents of the invention can be used to reduce a subject's body fat mass by at least 2% e.g. 3%, 4%, or 5%. 6%, 7%, 8%, 9%, 10%, 15% or more.

Alternatively, or in addition, treatment of obesity can be taken to include a reduction in a subject's BMI. In one embodiment, the subject's BMI can be reduced by less than 1 or more than 1, e.g. 2, 3, 4, 5, 6, 7, 8, 9, 10 kg/m² or greater.

In one embodiment, the agent lowers the concentration of total plasma cysteine (tCys). The agent may act on any target which is involved in the pathway that generates plasma cysteine. Without being bound by theory, the inventors' believe that cysteine increases fat mass by one or more of the following mechanisms, which can be categorised into two classes of mechanism: (a) local mechanisms in adipose tissue and (b) systemic mechanisms. The local mechanisms include for example, the inhibition of lipolysis, the stimulation of adipogenesis by increasing size of proliferation rate of adipocytes and/or adipocyte precursors and/or the enhancement of triglyceride accumulation in adipose tissue. Systemic mechanisms by which cysteine may increase the fat mass of a subject include for example, by decreasing metabolic rate and/or energy expenditure, enhancing the ability of the liver to package and secrete triglycerides to the plasma and/or altering the expression or protein levels of one or more of the following enzymes or proteins involved in lipid and energy metabolism:

i) Sterol regulatory element-binding protein

ii) Stearoyl CoA desaturase

iii) Monoacylglycerol glycerol acyltransferase

iv) Hydroxysteroid dehydrogenase

v) Acyl CoA synthetase

vi) Mitochondrial uncoupling proteins

vii) Lipid-droplet associated proteins.

Thus, the agent of the invention may act to inhibit cysteine activity on one or more of the mechanisms described above. In an embodiment, the agent may act to inhibit the cysteine action on tissues such as adipose tissue. A simplified pathway is shown in FIG. 1. In one embodiment, the agent modulates, e.g. reduces, plasma cystathionine concentration. In one embodiment, the agent modulates cystathionine beta-synthase function. For example, the agent may reduce the expression of cystathionine beta-synthase (CBS) or inhibit one or more of its biological functions. In one embodiment, the agent reduces the activity of the cystathionine beta-synthase so as to reduce cysteine levels in the plasma of a subject.

CBS is an enzyme that plays an essential role in homocysteine metabolism in eukaryotes. The CBS enzyme catalyses a pyridoxal 5′-phosphate ((PLP)-dependent condensation of homocysteine and serine to form cystathionine, which is then used to produce cysteine by another PLP-dependent enzyme, cystathionine γ lyase (CGL).

An agent which reduces the activity of CBS may act to decrease plasma cysteine and so reduce body fat mass in a subject and therefore may be useful in the treatment or prevention of obesity. The gene sequence of cystathionine beta synthase was first disclosed in Kraus J P et al (1998) Genomics 52: 312-324. The CBS gene contains a number of polymorphisms and the agent as described herein may act to reduce the activity of one or more of the cystathionine beta synthase variants.

In one embodiment, the agent modulates cystathionine γ-lyase (CGL) function, the rate-limiting step in cysteine formation. For example, the agent may reduce the expression of CGL or inhibit one or more of its biological functions. Drugs of the class exemplified by, but not limited to, propargylglycine are potent inhibitors of the enzyme's activity (Steegborn et al J Biol Chem 1999:274, 12675-12684) and drugs of the general group of non-steroidal anti-inflammatory agents (such as, but not limited to, indomethacin, ketoprofen and aspirin) reduce expression of the gene for CGL by inhibition of the ERK/Sp1 signalling pathway (Fiorucci et al Gastroenterology 2005: 129, 1210-1224).

In one embodiment, the agent modulates the formation of cysteine from cysteine-containing peptides by inhibiting dipeptidases e.g. renal membrane dipeptidase or dehydropeptidase. Examples of agents which may be suitable include, for example but not limited to, cilastatin. Cilastatin lowers plasma cysteine levels (Badiou et al. Clin Chem Lab Med 2005: 43, 332-334).

Other exemplary agents include those selected from: (a) inhibitors of enzymes involved in cysteine synthesis e.g. inhibitors of cystathionine beta-synthase or cysteine gamma lyase (=cystathionase); (b) inhibitors of dipeptidases that release cysteine from cysteinylglycine e.g. cilastatin; and (c) stimulators of cysteine turnover e.g. agents that stimulate the enzyme cysteine dioxygenase or the enzyme gamma glutamyl cysteine synthetase e.g. acetaminophen.

In one aspect of the invention, the agent is an agent that can decrease the active form of plasma cysteine e.g. by inhibiting release of cysteine from plasma protein-binding. In one embodiment, the agent may not reduce plasma cysteine concentration. In one embodiment, the agent may be an inhibitor of cellular uptake of cysteine e.g. sulfasalazine, which blocks the cysteine transporter. In one embodiment, the agent is an inhibitor of cellular action of cysteine e.g. the agent blocks the cysteine receptor e.g. by competitive inhibition. In one embodiment, the agent may be an inhibitor of cellular uptake of cystine which blocks the cystine transporter.

In one embodiment, the agent is for example mesna, which is currently used as an adjuvant in cancer therapies. This drug markedly reduces the plasma level of cysteine in humans, in a concentration-dependent manner and this action may be potentiated by concomitant administration of ifosamide (Smith et al J. Clin Pharmacol 2003: 43, 1324-1328).

In a further aspect of the present invention, there is provided a method of screening for an agent for the treatment of obesity comprising:

(a) administering a test agent to an animal; and
(b) detecting plasma concentration of one or more sulphur containing amino acids.

In one embodiment, the method comprises detecting plasma total cysteine concentration. The method may further comprise obtaining a sample from the animal, wherein optionally the sample is a blood sample and/or a plasma sample.

The method may further comprise repeating steps (a) and/or (b). The method may be for determining whether a test agent has anti-obesity effects and comprises obtaining a first weight of the animal prior to administration of the test agent and obtaining a second weight following administration of the test agent and comparing the first weight and the second weight.

In one embodiment, the method comprises obtaining a first measurement of body fat mass prior to administration of the test agent and obtaining a second measurement of body fat mass after administration of the test agent and comparing the first measurement and the second measurement. In one embodiment, the method is carried out on a non-human mammal.

The inventors for the first time have considered the causal link between cysteine levels and obesity. Previous studies have suggested that tCys changes as a result of a change in determinants such as BMI, cholesterol and diastolic blood pressure (El-Khairy et al, Clinical Chemistry 49:1, 113-120 (2003). There was no mention or suggestion in El-Khairy et al that tCys may have an effect on body fat levels. Furthermore, there was no suggestion that agents which reduce levels of tCys may be used to treat obesity nor of methods of treating obesity comprising manipulating tCys.

Other studies have taught away from the present invention and suggested the use of cysteine as a supplement to weight loss therapy. For example U.S. Pat. No. 7,268,161 suggests administering a nutritional supplement including cysteine in combination with a weight loss therapy that includes phentermine and/or diethylpropion with an SSRI medication, citalopram. There is no suggestion in U.S. Pat. No. 7,268,161 that an anti-cysteine agent may be used to treat obesity or prevent body fat mass increase.

Tozer et al, (Antioxid. Redox Signal 2008; 10: 395-402) have suggested a link between a diet containing a cysteine-rich protein and an increase in body weight. Tozer et al attributed the increase in body weight to a prevention of muscle wasting. There was no suggestion that the increase in body weight was as a result of increased body fat. Furthermore, there is no suggestion in Tozer et al to treat obesity using agents which inhibit cysteine activity.

In a further aspect of the invention, there is provided a method of encouraging weight gain in a subject comprising administrating an agent which increases cysteine activity and/or plasma cysteine levels.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

The invention will now be described by way of specific examples and with reference to the accompanying figures detailed below:

The following applies to all concentration-response curves (FIGS. 4,6,7,9,10):
1—Curves were plotted by Gaussian generalized additive regression models, as implemented in S-PLUS 6.2 for Windows (Insightful Corporation, Seattle, Wash.).
2—Solid lines represent the concentration-response curve and the shaded areas represent the 95% confidence intervals.
3—The reference value of zero on the Y-axis corresponds to the approximate value of the dependent (Y-axis) variable that is associated with the average value of the independent (X-axis) variable for all subjects. Positive values on the Y axis represent “greater than average values”, while negative values are “less than average”.
4—Curves are adjusted for age and gender.
5—P-values and partial correlation coefficients (partial r) were obtained by corresponding linear regression analyses
6—The lowest and highest 1 percentiles of the independent variables are not shown.

FIG. 1: Shows sulphur-containing amino acids-metabolic pathways. Cysteine: metabolic pathways and role of GGT in cysteine homeostasis. Located at cell membranes, GGT catalyzes breakdown of glutathione to glutamate and cysteinylglycine, which ultimately releases cysteine, in the gamma glutamyl cycle. Dotted arrows indicate pathways with omitted intermediates for purposes of clarity. CBS, cystathionine beta synthase; CGL, cystathionine gamma lyase; H2S, hydrogen sulfide; GGCS, gamma glutamyl cysteine synthase; CDO, cysteine dioxygenase.

A. Data from the Hordaland Homocysteine Study: n=5179 Norwegian Subjects.

FIG. 2: Table (Table 1) showing population characteristics from the Hordaland Homocysteine study. N=5179 Norwegian subjects. The footnotes to the table are as follows:

¹ Differences between the 4 age-gender groups were first tested by Chi Square test or ANOVA (p<0.001 for all variables) followed by group-wise comparisons with Bonferroni adjustment.
² Using log-transformed data
³ P<0.05 compared to middle aged men
⁴ P<0.05 compared to middle aged women
⁵ P<0.05 compared to elderly men

FIG. 3: Table (Table 2) showing body composition and anthropometric parameters by quintiles of tCys. The footnotes to the table are as follows:

¹ tCys, plasma total cysteine. Quintiles are age-group and gender-specific. Significance of difference between quintiles were tested by ANOVA (p,0.001 for all variables except height in men) followed by group-wise comparisons with Bonferroni adjustment using the lowest quintile as reference.
² p<0.05 compared to first quintile
³ p<0.001 compared to first quintile

FIG. 4: Association of plasma cysteine with body mass index, body total lean-mass and body total fat-mass, with reciprocal adjustment for lean mass or fat mass. Plasma cysteine showed a strong positive association with BMI and fat mass, but not with lean mass.

FIG. 5: Table (Table 3) showing estimated difference in body mass index, lean-mass (in kg) and fat-mass (in kg) at follow-up by quintiles of change in tCys and tHcy over 6 years. The footnotes to the table are as follows:

¹ tCys, plasma total cysteine; tHcy, plasma total homocysteine. The models were calculated by linear regression and estimated the difference in mean BMI, fat-mass or lean-mass between each quintile and the reference quintile (lowest quintile) of change in tCys or tHcy.
² Model 1, adjusted for age, gender, baseline BMI, baseline tCys, fat-mass in case of lean-mass, and lean-mass in case of fat-mass.
³ Model 2, adjusted for all model 1 variables+changes in plasma total cholesterol and triacylglycerol, change in smoking habits and systolic blood pressure+plasma creatinine and physical activity at follow-up.
⁴ Model 3, adjusted for age, gender, baseline BMI, baseline tHcy, fat-mass in case of lean-mass, and lean-mass in case of fat-mass
⁵ Model 4, adjusted for all Model 3 variables+changes in plasma concentrations of tCys, vitamin B12, folate, triacylglycerol and cholesterol, changing in smoking habits, and plasma creatinine and physical activity at follow-up.

FIG. 6: Association of percent change in plasma cysteine during a 6-year follow-up period with body total fat-mass and lean-mass at follow-up, with adjustment for baseline plasma cysteine and baseline BMI, and reciprocal adjustment for lean mass or fat mass. Subjects whose plasma cysteine decreased by 10% had a fat mass at follow-up that was ˜2 kg lower than subjects who had no change in plasma cysteine (p<0.001). Changes in plasma cysteine had no effect on lean mass.

C and D—are additionally adjusted for changes in smoking habits, plasma triglycerides and total cholesterol+physical activity, blood pressure and plasma creatinine and at follow-up.

FIG. 7: Association of plasma cysteine at baseline with body total fat mass 6 years later. B—Adjusted for lean mass at follow-up. C— Adjusted for lean mass, at follow-up+baseline concentrations of triglycerides, cholesterol, homocysteine folate and vitamin B12. Subjects with the highest cysteine at baseline had a fat mass at follow up which was approximately 11 kg higher compared to those with lowest cysteine levels. Variations in lean mass and plasma concentrations of triglycerides and cholesterol were taken into account.

B—Data from the COMAC Cohort: n=1550 Subjects from 9 European Countries.

FIG. 8: Table (Table 4) showing selected characteristics of the study population according to case-control status and gender. The footnote to the table is as follows: ¹ Data presented as median (interquartile range)

FIG. 9: Association of plasma gamma glutamyl transferase with plasma total cysteine, adjusted for case-control status, plasma total homocysteine and creatinine, and cysteinylglycine.

FIG. 10: Associations of plasma total cysteine (tCys) and gamma glutamyl transferase (GGT) with BMI, adjusted for case-control status. B and D additionally adjusted for systolic and diastolic blood pressure and smoking habits and the following plasma/serum variables: GGT or tCys, triacyl glycerol, HDL and LDL cholesterol, cysteinylglycine, homocysteine, creatinine, urea and glutamic oxalacetic transferase. Plasma cysteine was more strongly associated with BMI than GGT, and the cysteine-BMI association was independent of GGT and other factors.

FIG. 11: is a table (Table 5) showing the odds ratio for obesity by quartiles of plasma tCys(a). The table includes the following:

(a) tCys, total cysteine; Obesity is defined as BMI;::30; n=1461-1550 men and women.
(b) Adjusted for age, gender and case-control status.
(c) Adjusted for model 1 variables and GGT.
(d) Adjusted for model 2 variables+plasma/serum concentrations of total homocysteine, creatinine, glutamic oxalacetic transferase and triglycerides+smoking habits and diastolic blood pressure.
c—Data from Cysteine-Lowering Experiments in Rats

FIG. 12: A graph showing the percent change of median plasma sulfur amino acid concentrations and body weight parameters in experimental rats relative to control. All differences are significant at p:50.004 by Mann-Whitney U test. Abbreviations: tCys, total cysteine; Met, methionine; tHcy, total homocysteine.

D—Data from Study of Patients Undergoing Weight Loss Surgery

FIG. 13. Mean BMI and tCys before and 6 months after bariatric surgery (N=60) compared to a control group matched in age and gender (N=60). The shaded area shows the interquartile ranges of tCys and BMI in the Hordaland Study (HHS), and the line shows the tCys-BMI-dose-response relationship in HHS. The length of the X-axis represents tCys references limits in HHS. In the extremely obese patients the change in tCys after surgery is negligible compared to the dramatic reduction (27%) in BMI.

FIG. 14: A flow diagram showing an experimental design to test the effect of cysteine reduction on fat mass.

### DETAILED DESCRIPTION

The present invention relates to the modulation of body fat mass cornpnsmq modulating plasma levels of a sulphur-containing amino acid e.g. cysteine. The present invention comprises the use of an agent which modulates, e.g. reduces, the level of the sulphur containing amino acid in plasma for the modulation of body fat mass level of a subject. In one embodiment, the agent is for the treatment or prevention of obesity. In one embodiment, the agent is for the reduction of body fat mass in an overweight subject. In one embodiment, the agent reduces the level of plasma levels of cysteine. In an embodiment, the agent reduces the effect of cysteine on adipose tissue.

Certain embodiments of the present invention are concerned with the prevention of weight gain in a subject. In one embodiment, the agent is for the prevention of an increase in body fat mass, in particular but not exclusively in subjects for whom an increase in body fat mass would be associated with potentially serious health risks. Such subjects include for example individuals suffering from or are believed to be at risk from suffering from disorders including, for example, cardiovascular disease, diabetes mellitus and certain types of cancer.

In one embodiment, the subject has an elevated plasma cysteine level. In one embodiment the subject is not obese but has high risk of developing obesity secondary to a high cysteine level. In one embodiment, a subject is considered to have a high cysteine level if their plasma cysteine level is in the highest quartile for their age and gender.

Patients who are at risk of developing atherosclerotic lesions and therefore who may benefit from the prevention of weight gain due to an increase in body fat mass can be identified using risk assessment calculations. Such risk assessment calculations may include the PROCAM coronary heart disease risk function and the Framingham coronary heart disease risk function. The PROCAM risk function estimates the probability of developing coronary death or first myocardial infarction within ten years and employs age, systolic blood pressure, LDL and HDL cholesterol, triglycerides, cigarette use, diabetes and family history of myocardial infarction as risk factors.

The FRAMINGHAM risk function estimates the probability of developing coronary death, myocardial infarction (recognised and unrecognised), angina pectoris or coronary insufficiency (total CHD end points) within ten years, taking age, blood pressure, LDL and HDL cholesterol, cigarette use and diabetes as risk factors. (Anderson K M et al, Circulation 1991; 83:356-362). Thus, agents of the present disclosure may be used in the treatment, particularly chronic or long-term treatment, of patients who have been identified as having a risk factor of 45 or more. Thus, agents of the present invention may be administered to patients who have been identified as having a PROCAM score of 45, 46, 47, 48, 49, 50, 51, 52, 53, 54, 55, 56, 57, 58, 59 or 60 in order to prevent or reduce weight gain. Such treatment is prophylactic.

Methods, uses and agents of the present disclosure can be utilised for or in the treatment of patients who are at risk of development of atherosclerotic plaques. Such patients may have been identified as having some or all of the risk factors associated with atherosclerosis. Thus, one class of patients which could be treated using agents, methods and uses of the present invention are patients who have been identified using the Framingham risk factor as having a defined level of risk of developing atherosclerotic lesions and complications thereof and for which body fat mass gain may be disadvantageous. Thus, the defined level of risk may be determined according to the points obtained by the patient using the Framingham method. Thus, in one embodiment of the present invention, there is provided a method of treating a male patient who has been identified as having a points score according to the Framingham study of 11 or more. Alternatively, when the patient is a female subject, the patient may have been identified as having a risk factor, using the Framingham study, wherein the patient has been awarded 20 or more points according to the Framingham point scores.

In another embodiment the agent acts to decrease the active form of cysteine in plasma, e.g. it prevents cysteine release from protein binding or prevents reduction of cystine (disulfide) to active free cysteine.

In a further embodiment the agent enhances conversion of cysteine to other natural substrates as glutathione or taurine. Examples include but are not restricted to compounds, including sulphur amino acids, that stimulate the enzyme cysteine dioxygenase, which catalyses the first step of the conversion of cysteine to the amino acid taurine (Deborah L. Bella, Christine Hahn, and Martha H. Stipanuk. Am J Physiol Endocrinol Metab 1999; 277 (1): E144-E153). Other examples include agents that enhance incorporation of cysteine into glutathione e.g. acetaminophen. (Lauterburg B H, Mitchell J R. J. Hepatol. 1987 April; 4 (2): 206-11.).

In one embodiment, the agent binds to one or more cysteine receptors or one or more cysteine transporters to block their action e.g. by preventing cysteine binding to a receptor or transporter. Exemplary agents in this class include for example sulfasalazine, which inhibits cellular uptake of cysteine via the cystine transporter xc-. Other examples in this class include L-homocysteate, ibotenate, L-serine-O-sulphate, quisqualate, (RS)-4-bromohomoibotenate, and (S)-4-carboxyphenylglycine. (Neuropharmacology Volume 46, Issue 2, February 2004, Pages 273-284 Sarjubhai A. Patel, et al).

In another embodiment the invention includes inducing weight loss or preventing weight gain by reducing dietary intake of cysteine and/or methionine i.e. by restricting the ingestion of foods rich in cysteine and methionine. Cysteine-rich foods include onions, garlic, eggs, cabbage and broccoli. (Katherine D. et al., Journal of the American Dietetic Association, 1996: 96(1); 46-48). Methionine is abundant in animal protein, including meat, chicken and fish.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Definitions and additional information known to one of skill in the art in immunology can be found, for example, in Fundamental Immunology, W. E. Paul, ed., fourth edition, Lippincott-Raven Publishers, 1999.

### Exemplary Agents

Exemplary agents include, but are not limited to, proteins, peptides, antibodies, peptibodies, carbohydrates and small organic molecules. Further details of suitable agents are provided below:

In one embodiment, the agent is an isolated protein, peptide, antibody, antibody fragment or fusion protein. An “isolated” or “purified” protein or biologically active fragment thereof is substantially free of cellular material or other contaminating proteins from the cell or tissue source from which the protein is derived, or substantially free of chemical precursors or other chemicals when chemically synthesized. The language “substantially free of cellular material” includes preparations of the protein in which the protein is separated from cellular components of the cells from which it is isolated or recombinantly produced.

In all of the embodiments of the invention described herein in which the agent is a polypeptide, the amino acid sequence of the agent may be modified by one or more changes in sequence which do not eliminate the underlying biological function and utility of the agents as described herein. Modifications may include substitution of individual amino acids with other naturally occurring or non-naturally occurring amino acids.

The agents of the invention may be, for example, an antibody or fragment thereof, e.g. a Fab fragment. The antibody may be for example an antibody which binds to cysteine or the cysteine receptor or cysteine transporter. Preferred antibodies and fragments are Fab fragments or scFv. Naturally within the scope of the agents of the invention are antibodies or fragments which are monoclonal, polyclonal, chimeric, human, or humanized. In one embodiment, the agent of the present invention is an antibody. An antibody and immunologically active portions thereof, for instance, are typically molecules that contain an antigen binding site which specifically binds (immunoreacts with) an antigen.

In one embodiment the agent is an antibody which binds to one or more cysteine receptors or one or more cysteine transporters to block their action e.g. by preventing cysteine binding to a receptor or transporter.

A naturally occurring antibody (for example, IgG) includes four polypeptide chains, two heavy (H) chains and two light (L) chains interconnected by disulfide bonds. The two heavy chains are linked to each other by disulfide bonds and each heavy chain is linked to a light chain by a disulfide bond. There are two types of light chain, lambda (A) and kappa (k). There are five main heavy chain classes (or isotypes) which determine the functional activity of an antibody molecule: IgM, IgD, IgG, IgA and IgE. Full-length immunoglobulin light chains are generally about 25 Kd or 214 amino acids in length. Full-length immunoglobulin heavy chains are generally about 50 Kd or 446 amino acid in length. Light chains are encoded by a variable region gene at the NH2-terminus (about 110 amino acids in length) and a kappa or lambda constant region gene at the COOH—terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al., Eur. J. Immunol. 17:105, 1987; Huston et al., Proc. Natl. Acad. Sci. U.S.A., 85:5879-5883, 1988; Bird et al., Science 242:423-426, 1988; Hood et al., Immunology, Benjamin, N.Y., 2nd ed., 1984; Hunkapiller and Hood, Nature 323:15-16, 1986).

Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, transplacental mobility, complement binding, and binding to Fc receptors. An immunoglobulin light or heavy chain variable region includes a framework region interrupted by three hypervariable regions, also called complementarity determining regions (CDR's) (see, Sequences of Proteins of Immunological Interest, E. Kabat et al., U.S. Department of Health and Human Services, 1983). As noted above, the CDRs are primarily responsible for binding to an epitope of an antigen. The specificity of the antibody resides in the structural complementarity between the antibody combining site and the antigenic determinant.

In one embodiment, the antibody is a monoclonal antibody. A monoclonal antibody is produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Generally, a monoclonal antibody is produced by a specific hybridoma cell, or a progeny of the hybridoma cell propagated in culture. A hybridoma or other cell producing an antibody may be subject to genetic mutation or other changes, which may or may not alter the binding specificity of antibodies produced.

A suitable class of agents may be chimeric antibodies which bind to cysteine e.g. in its disulphide form, cystine, or a member of the cysteine synthesis pathway. Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715, which is herein incorporated by reference.

In one embodiment, the agent may be a humanized antibody or fragment thereof. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Exemplary conservative substitutions are those such as gly, ala; val, ile, leu; asp, glu; asn, gln; ser, thr; lys, arg; and phe, tyr (see U.S. Pat. No. 5,585,089, which is incorporated herein by reference). Humanized immunoglobulins can be constructed by means of genetic engineering, e.g., see U.S. Pat. No. 5,225,539 and U.S. Pat. No. 5,585,089, which are herein incorporated by reference.

In one embodiment, the agent is a human antibody. A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g., Dower et al., PCT Publication No. WO91/17271; McCafferty et al., PCT Publication No. WO92/001047; and Winter, PCT Publication No. WO92/20791, which are herein incorporated by reference), or selected from a human combinatorial monoclonal antibody library (see the Morphosys website). Human antibodies can also be prepared by using transgenic animals carrying a human immunoglobulin gene (e.g., see Lonberg et al., PCT Publication No. WO93/12227; and Kucherlapati, PCT Publication No. WO91/10741, which are herein incorporated by reference).

Antibodies may also be obtained using phage display technology. Phage display technology is known in the art for example Marks et al J. Mol. Biol. 222: 581-597 and Ckackson et al, Nature 352: 624-628, both incorporated herein by reference. Phage display technology can also be used to increase the affinity of an antibody. To increase antibody affinity, the antibody sequence is diversified, a phage antibody library is constructed, and a higher affinity binders are selected on antigen (see for example Marks et al Bio/Technology 10:779-783, Barbas et al Proc. Natl. Acad. Sci. USA 91:3809-3813 and Schier et al J. Mol. Biol. 263: 551-567, all incorporated herein by reference.

In one embodiment, the agent is an antibody fragment. Various fragments of antibodies have been defined, including Fab, (Fab)₂, Fv, dsFV and single-chain Fv (scFv) which have specific antigen binding. These antibody fragments are defined as follows: (1) Fab, the fragment that contains a monovalent antigen-binding fragment of an antibody molecule produced by digestion of whole antibody with the enzyme papain to yield an intact light chain and a portion of one heavy chain or equivalently by genetic engineering; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction or equivalently by genetic engineering; (4) F(Ab′)₂, a dimer of two FAb′ fragments held together by disulfide bonds; (5) Fv, a genetically engineered fragment containing the variable region of the light chain and the variable region of the heavy chain expressed as two chains; dsFV, which is the variable region of the light chain and the variable region of the heavy chain linked by disulfide bonds and (6) single chain antibody (“SCA”), a genetically engineered molecule containing the variable region of the light chain, the variable region of the heavy chain, linked by a suitable polypeptide linker as a genetically fused single chain molecule. Single chain antibodies may also be referred to as single chain variable fragments (scFv). Methods of making these fragments are routine in the art.

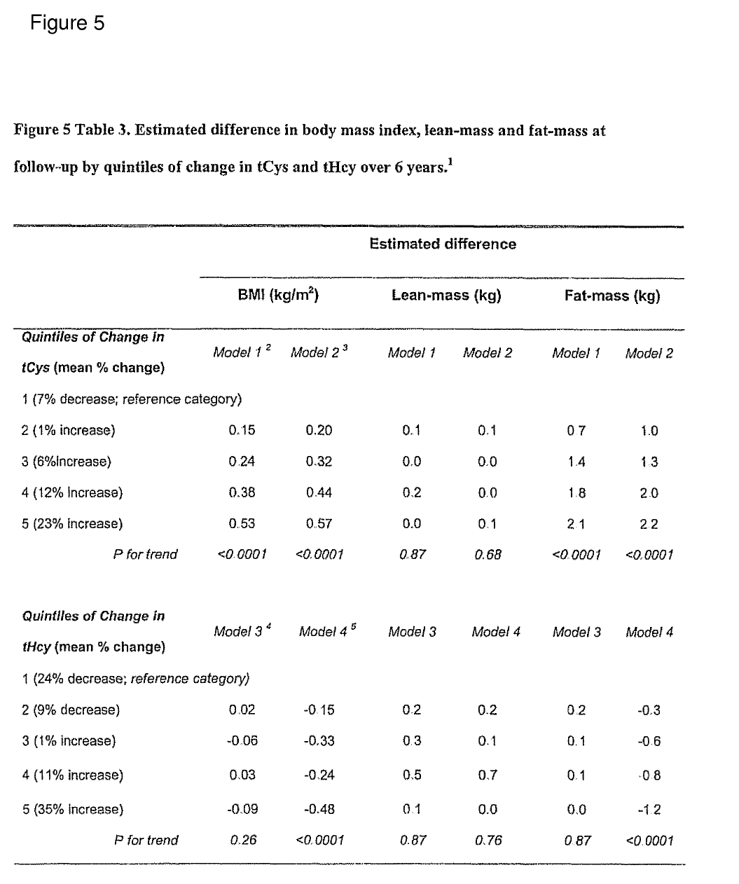

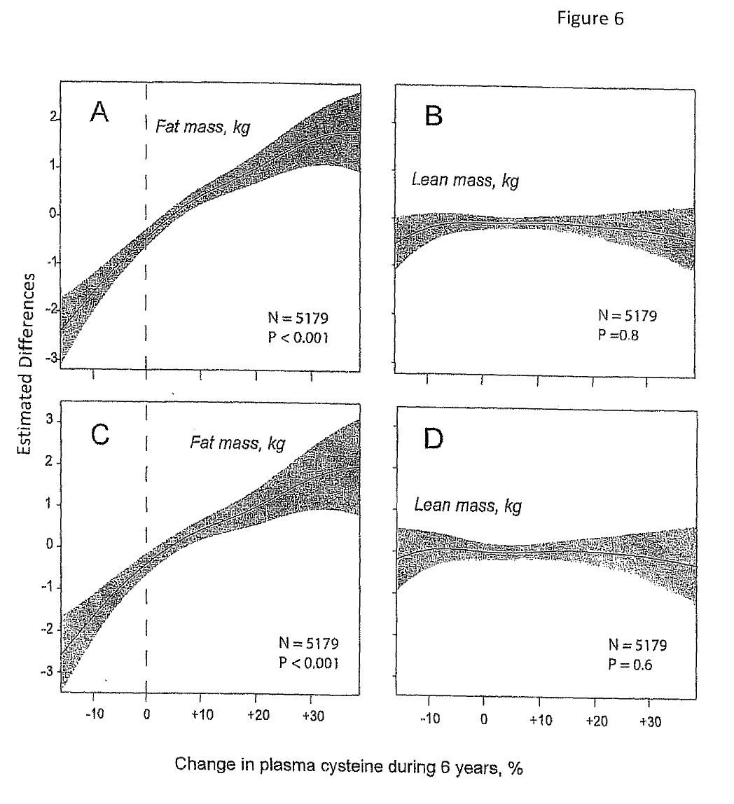

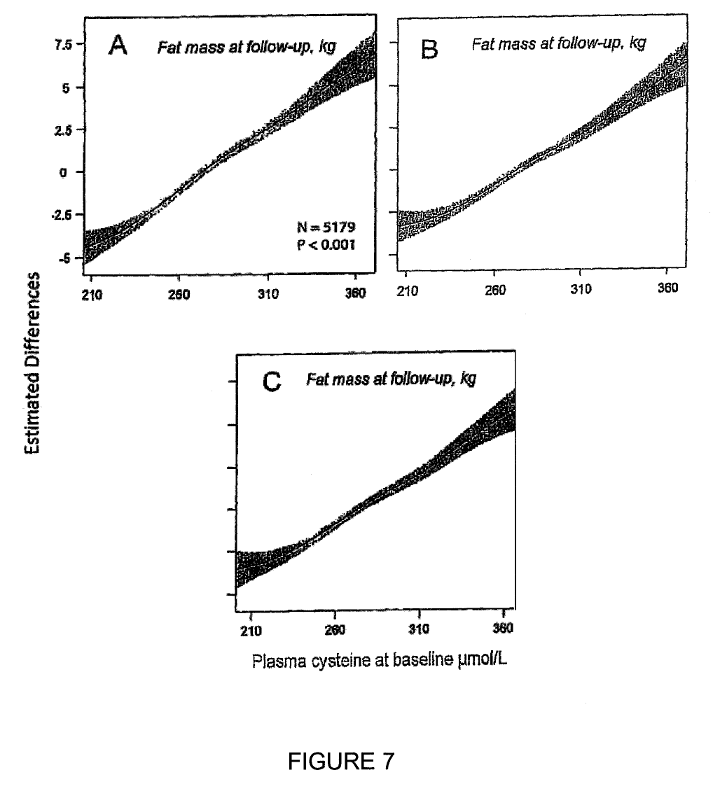

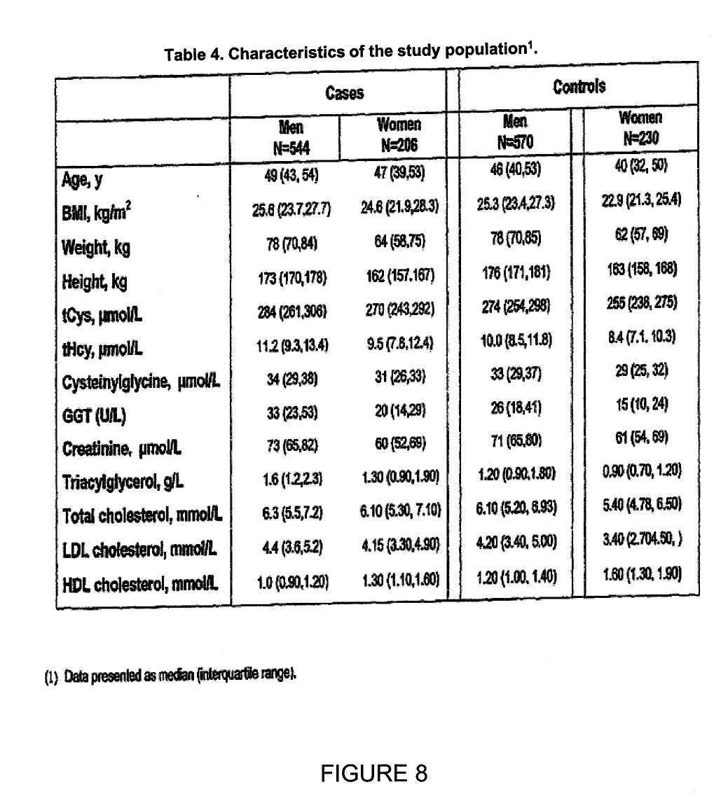

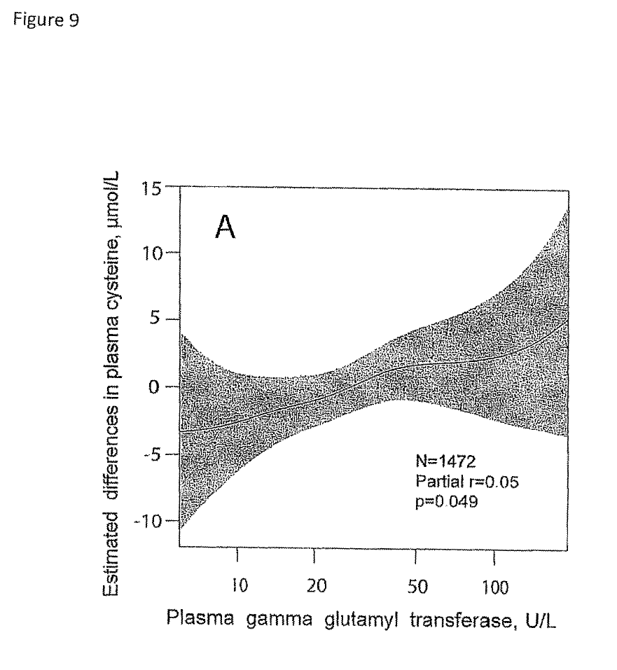

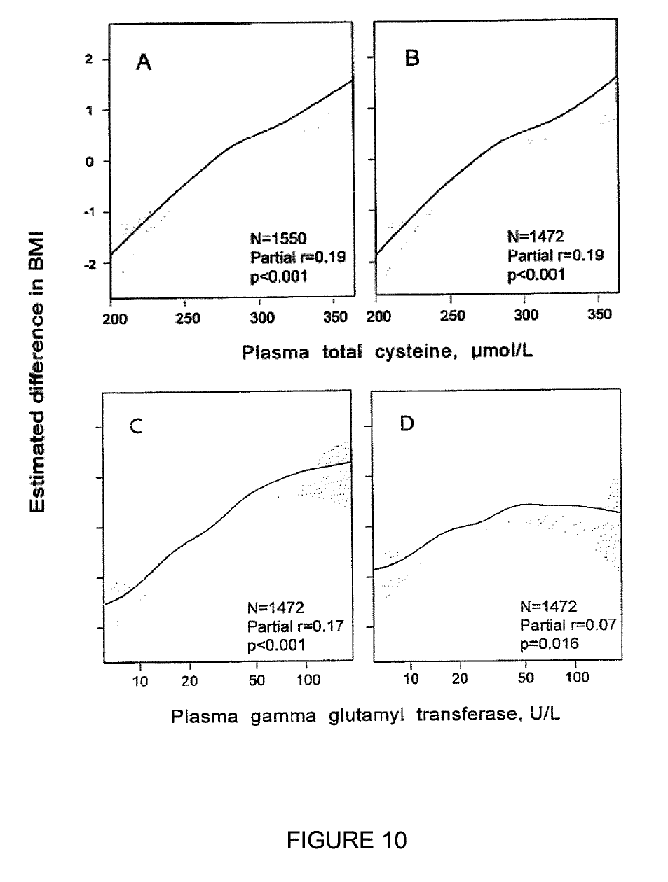

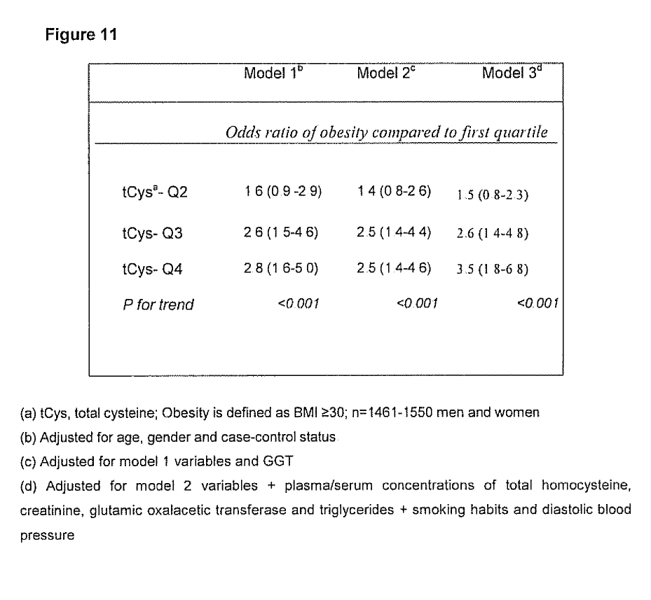

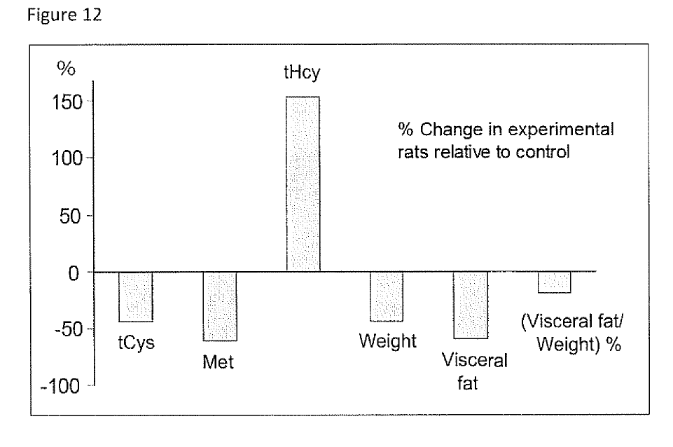

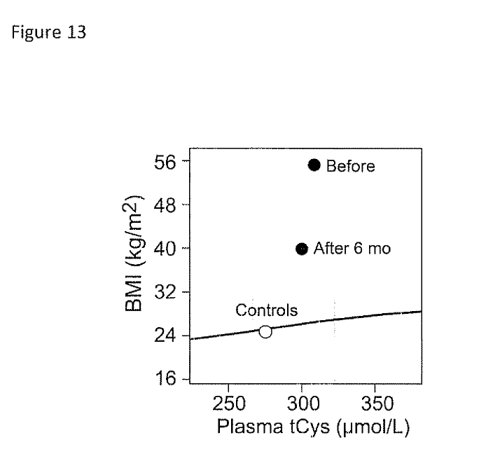

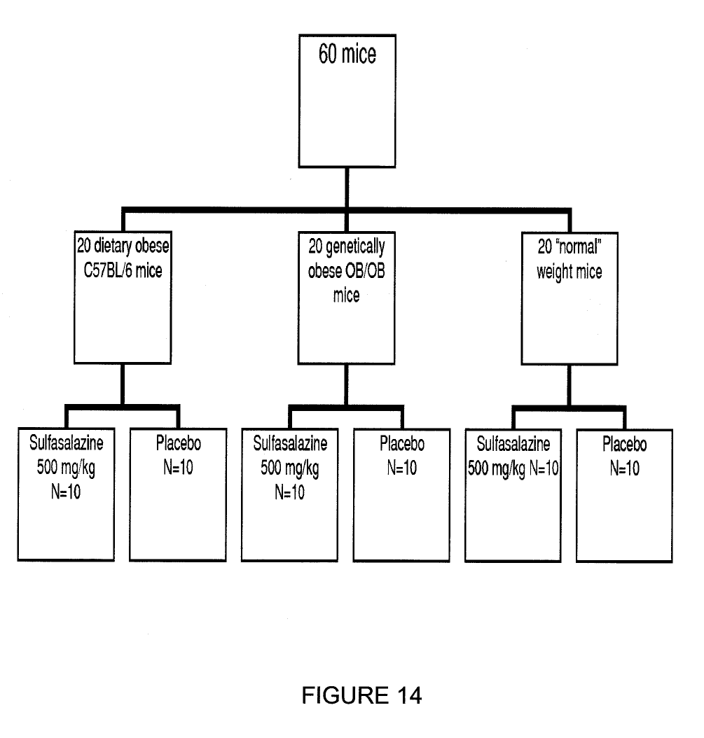

Reference is made to the numbering scheme from Kabat, E. A., et al., Sequences of Proteins of Immunological Interest (National Institutes of Health, Bethesda, Md. (1987) and (1991). In these compendiums, Kabat lists many amino acid sequences for antibodies for each subclass, and lists the most commonly occurring amino acid for each residue position in that subclass. Kabat uses a method for assigning a residue number to each amino acid in a listed sequence, and this method for assigning residue numbers has become standard in the field. For purposes of this invention, to assign residue numbers to a candidate antibody amino acid sequence which is not included in the Kabat compendium, one follows the following steps. Generally, the candidate sequence is aligned with any immunoglobulin sequence or any consensus sequence in Kabat. Alignment may be done by hand, or by computer using commonly accepted computer programs; an example of such a program is the Align 2 program discussed in this description. Alignment may be facilitated by using some amino acid residues which are common to most Fab sequences. For example, the light and heavy chains each typically have two cysteines which have the same residue numbers; in VL domain the two cysteines are typically at residue numbers 23 and 88, and in the VH domain the two cysteine residues are typically numbered 22 and 92. Framework residues generally, but not always, have approximately the same number of residues, however the CDRs will vary in size. For example, in the case of a CDR from a candidate sequence which is longer than the CDR in the sequence in Kabat to which it is aligned, typically suffixes are added to the residue number to indicate the insertion of additional residues (see, e.g. residues 100abcde in FIG. 5). For candidate sequences which, for example, align with a Kabat sequence for residues 34 and 36 but have no residue between them to align with residue 35, the number 35 is simply not assigned to a residue.

CDR and FR residues are also determined according to a structural definition (as in Chothia and Lesk, J. Mol. Biol. 196:901-917 (1987). Where these two methods result in slightly different identifications of a CDR, the structural definition is preferred, but the residues identified by the sequence definition method are considered important FR residues for determination of which framework residues to import into a consensus sequence.

In one embodiment, the agent is an antibody which binds to cysteine. In a further embodiment, the agent is an antibody as described above which binds to the cystathionine beta-synthase enzyme so as to block its activity and reduce cysteine production. In a further embodiment, the agent is an antibody as described above which binds to the cystathionine γ-lyase (CGL) enzyme so as to block its activity and reduce cysteine production.

### Aptamers

A further class of agents useful in the present invention are aptamers e.g. aptamers which bind to cysteine. Aptamers have been defined as artificial nucleic acid ligands that can be generated against amino acids, drugs, proteins and other molecules. They are isolated from complex libraries of synthetic nucleic acids by an iterative process of adsorption, recovery and re-amplification.

RNA aptamers are nucleic acid molecules with affinities for specific target molecules. They have been likened to antibodies because of their ligand binding properties. They may be considered as useful agents for a variety of reasons. Specifically, they are soluble in a wide variety of solution conditions and concentrations, and their binding specificities are largely undisturbed by reagents such as detergents and other mild denaturants. Moreover, they are relatively cheap to isolate and produce. They may also readily be modified to generate species with improved properties. Extensive studies show that nucleic acids are largely non-toxic and non-immunogenic and aptamers have already found clinical application. Furthermore, it is known how to modulate the activities of aptamers in biological samples by the production of inactive dsRNA molecules in the presence of complementary RNA single strands (Rusconi et al., 2002).

It is known from the prior art how to isolate aptamers from degenerate sequence pools by repeated cycles of binding, sieving and amplification. Such methods are described in U.S. Pat. No. 5,475,096, U.S. Pat. No. 5,270,163 and EP0533 38 and typically are referred to as SELEX (Systematic Evolution of Ligands by EX-ponential Enrichment). The basic SELEX system has been modified for example by using Photo-SELEX where aptamers contain photo-reactive groups capable of binding and/or photo cross-linking to and/or photo-activating or inactivating a target molecule. Other modifications include Chimeric-SELEX, Blended-SELEX, Counter-SELEX, Solution-SELEX, Chemi-SELEX, Tissue-SELEX and Transcription-free SELEX which describes a method for ligating random fragments of RNA bound to a DNA template to form the oligonucleotide library. However, these methods even though producing enriched ligand-binding nucleic acid molecules, still produce unstable products. In order to overcome the problem of stability it is known to create enantiomeric “spiegelmers” (WO 01/92566). The process involves initially creating a chemical mirror image of the target, then selecting aptamers to this mirror image and finally creating a chemical mirror image of the SELEX selected aptamer. By selecting natural RNAs, based on D-ribose sugar units, against the non-natural enantiomer of the eventual target molecule, for example a peptide made of D-amino acids, a spiegelmer directed against the natural L-amino acid target can be created. Once tight binding aptamers to the non-natural enantiomer target are isolated and sequenced, the Laws of Molecular Symmetry mean that RNAs synthesised chemically based on L-ribose sugars will bind the natural target, that is to say the mirror image of the selection target. This process is conveniently referred to as reflection-selection or mirror selection and the L-ribose species produced are significantly more stable in biological environments because they are less susceptible to normal enzymatic cleavage, i.e. they are nuclease resistant.

In one embodiment, the agent is an aptamer that binds to cysteine e.g. plasma cysteine after chemical reduction, or to cystine, or to cysteine-mixed disulfides, including cysteine bound to a particular protein via a disulfide bond. In one embodiment, the agent is an aptamer that binds to a cystathionine beta-synthase (CBS) gene or to the cystathionine beta-synthase (CBS) gene product. In one embodiment, the agent is an aptamer that binds to a cystathionine γ-lyase (CGL) gene or to the cystathionine γ-lyase (CGL) gene product.

### Proteins and Peptides

In one embodiment, the agent is a peptide or polypeptide. In one embodiment, the agent is a peptibody. The term “peptibody” refers to a molecule comprising an antibody Fc domain attached to at least one peptide. The production of peptibodies is generally described in PCT publication WO 00/24782, published May 4, 2000.

In one embodiment, the agent is a fusion protein i.e. a protein comprising at least two heterologous peptide sequences. The fusion protein may comprise a linker between the at least two peptide sequences. In one embodiment, the fusion protein is an antibody fusion protein. Examples of antibody fusion proteins are detailed in “Antibody Fusion Proteins” (Chamow and Ashenazi, Wiley-Liss 1999). In one embodiment, the agent may be an Fc fusion protein i.e. comprises an Fc portion of an antibody.

The agents of the present invention, if comprising a peptide sequence, for example an antibody, a fusion protein, a peptide or a protein, may be encoded by a nucleic acid sequence. The present invention includes any nucleic acid sequence which encodes an agent as defined herein. The present invention also includes a nucleic acid sequence which encodes the agent of the invention but which differs from the wild-type nucleic acid as a result of the degeneracy of the genetic code.

The present invention also includes nucleic acids that share at least 80% homology with a nucleic acid sequence which encodes an agent of the present invention. In particular, the nucleic acid may have 80%, 85%, 90%, 91%, 92%, 93%, 94%, 95%, 96%, 97%, 98% or 99% homology to a nucleic acid which encodes an agent of the present invention.

Calculations of sequence homology or identity (the terms are used interchangeably herein) between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In one embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

In one aspect of the invention, there is provided a nucleic acid molecule which hybridises under stringent conditions to a nucleic acid molecule which encodes an agent of the present invention. Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following have been found as exemplary for hybridization conditions but without limitation:

Very High Stringency (Allows Sequences that Share at Least 90% Identity to Hybridize)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Allows Sequences that Share at Least 80% Identity to Hybridize)

Hybridization: 5×−6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Allows Sequences that Share at Least 50% Identity to Hybridize)

Hybridization: 6×SSC at RT to 55° C. for 16-20 hours

Wash at least twice: 2×-3×SSC at RT to 55° C. for 20-30 minutes each.

In one embodiment, the nucleic acids hybridize over substantially their entire length.

### Uses of Agents

The agents as described herein may be used to treat an overweight or obese subject. The agents may also be used to reduce fat mass levels in a subject. In one embodiment, the agents may be used to prevent an increase in fat mass level in a subject with normal fat mass. In one embodiment, the agents may be used to prevent an increase in fat mass level in a subject for whom an increase in fat mass would represent a significant health risk.

Actual dosage levels of active ingredients in the pharmaceutical compositions of this disclosure may be varied so as to obtain an amount of the active agent(s) that is effective to achieve the desired therapeutic response for a particular patient, composition, and mode of administration (referred to herein as a “therapeutically effective amount”). The selected dosage level will depend upon the activity of the particular agent, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

Also included in the present invention is a pharmaceutical formulation comprising an agent as described herein; in embodiments the formulation is a composition comprising the agent and a pharmaceutically acceptable diluent, carrier or excipient. Such formulations may further routinely contain pharmaceutically acceptable concentrations of salt, buffering agents, preservatives, compatible carriers, supplementary immune potentiating agents such as adjuvants and cytokines and optionally other therapeutic agents.

The formulations may also include antioxidants and/or preservatives. As antioxidants may be mentioned tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. The pharmaceutically acceptable carriers useful in the methods disclosed herein are conventional. Remington's Pharmaceutical Sciences, by E. W. Martin, Mack Publishing Co, Easton, Pa., 15th Edition (1975), describes compositions and formulations suitable for pharmaceutical delivery of the agents herein disclosed.

The present inventive method includes the administration to an animal, such as a mammal, particularly a human, in need of prevention of an increase in body fat mass, of an effective amount, e.g., a therapeutically effective amount, of one or more of the aforementioned present agents, alone or in combination with one or more other pharmaceutically active agents.

The present inventive method includes the administration to an obese animal, such as a mammal, particularly a human an amount e.g., a therapeutically effective amount, of one or more of the aforementioned present inventive agents, alone or in combination with one or more other pharmaceutically active agents.

The present inventive method includes the administration to an animal, such as a mammal, particularly a human, in need of reduction of body fat mass, of an effective amount, e.g., a therapeutically effective amount, of one or more of the aforementioned present inventive agents, alone or in combination with one or more other pharmaceutically active agents.

The present invention also provides a product for reducing cysteine activity or uptake in a subject. In one embodiment, the product includes an agent as described herein. In one embodiment, the product is a nutraceutical. In an embodiment, the product is a meal replacement product. Such meal replacement products may be in the form of a powder which is suspendable in a liquid. Alternatively, the meal replacement product may be a liquid or a solid. Meal replacement products typically comprise a number of elements to ensure a subject receives additional nutrition even if on a low calorie diet. Thus, the product may include for example vitamins and minerals, a protein source, a fat source, a carbohydrate source and trace elements.

In one embodiment, the product may comprise an isolated soy protein as a protein source. In one embodiment, the carbohydrate source may be glucose, fructose and/or maltodextrine.

### Delivery of Active Agents

The agent of the present invention may be delivered to the subject by any suitable means. The skilled reader will appreciate that the administration may take place periodically throughout the term of the treatment, e.g. at periods of twice a day, once a day or longer. Substantially continuous administration by, for example, infusion is not excluded. In one embodiment, the mode of administration of the agent of the invention may be intravenous, inter-arterial or subcutaneous injection or infusion, or by oral administration.

In one embodiment, the agent is for oral administration. According to a further aspect of the disclosure there is provided an oral pharmaceutical formulation including an agent of the disclosure, in admixture with a pharmaceutically acceptable adjuvant, diluent or carrier.

The oral pharmaceutical formulation may be for repeated administration e.g. one a day, two a day or greater frequency. Solid dosage forms for oral administration include capsules, tablets (also called pills), powders and granules. In such solid dosage forms, the active compound is typically mixed with at least one inert, pharmaceutically acceptable excipient or carrier such as sodium citrate or dicalcium phosphate and/or one or more fillers, extenders, humectants, dissolution aids, ionic surface active agents. The active compounds may also be in micro-encapsulated form, if appropriate, with one or more of excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers.

The agents may be for administration via parental route. Parenteral preparations can be administered by one or more routes, such as intravenous, subcutaneous, intradermal and infusion; a particular example is intravenous. A formulation disclosed herein may be administered using a syringe, injector, plunger for solid formulations, pump, or any other device recognized in the art for parenteral administration.

### Screening Assays

In one aspect of the present invention, there is provided a method for predicting the occurrence of obesity in a subject or a group of subjects by screening the subject(s) for the presence of high cysteine levels.

In one embodiment, the method comprises treating or preventing obesity or fat mass increase by decreasing activity or plasma concentrations of plasma cysteine in a subject identified as having high cysteine levels. In one embodiment, the method comprises administering an agent as described herein to the subject. In one embodiment, the subject is considered to have high cysteine levels if their plasma cysteine is the highest quartile for their age and gender group.

The invention provides a method (also referred to herein as a “screening assay”) for identifying modulators, i.e., candidate or test compounds or agents (e.g., peptides, peptidomimetics, small molecules or other drugs) which act to inhibit or reduce sulphur-containing amino acid activity. In one embodiment, the method is for identifying modulators which inhibit cysteine activity on adipocytes and adipose tissue. In one embodiment, the test compound or agent is tested for its effect on plasma cysteine concentration. In one embodiment, the method is for screening for agents which reduce obesity in a subject.

In one embodiment, the invention provides assays for screening candidate or test compounds or diets which inhibits, e.g. reduces, cysteine activity. The assay may be for screening for compounds or diets which reduces plasma concentration of cysteine (tCys). The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In one embodiment, the method comprises administering a test compound to an animal and detecting the effect of the test compound on the plasma cysteine concentration. In one embodiment, the plasma cysteine concentration is measured as plasma total cysteine (tCys). In one embodiment, the method comprises detecting the effect of the test compound on adipose tissue. In one embodiment, the method comprises obtaining a sample from the animal. In one embodiment, the sample comprises a blood sample and/or a plasma sample. In one embodiment, the method comprises carrying out HPLC (High Performance Liquid Chromatography) on the sample. In one embodiment, the method comprises carrying out liquid chromatography-electrospray tandem mass spectrometry (LC-MS/MS) on the sample. In one embodiment, the method comprises carrying out gas chromatography mass spectrometry (GC-MS) on the sample. In one embodiment, the method comprises adding a reductant to the sample.

In one embodiment, the method comprises repeated administration of the test compound to the animal. In one embodiment, the method comprises formulating the test compound into a product for treating obesity or prevent weight gain in a subject. The test compound may reduce the tCys concentration. In an embodiment, the animal is a mammal e.g. a non-human mammal.

In one embodiment, the method comprises detecting the tCys levels using any method detailed in Chwatko and Bald, Talanta, Vol. 52., Issue 3, 2000 p 509-515. Other methods of determining tCys levels in samples included e.g. Liquid chromatography (LC), HPLC, capillary electrophoresis (CE) and gas chromatography (GC). In one embodiment, the method comprises determining cysteine concentrations using the method disclosed in Krijt J, Vackova M, Kozich V, Clin Chem. 2001 October; 47(10):1821-8. In one embodiment, the method comprises determining cysteine concentration using the method disclosed in Fiskerstrand T et al Clin Chem. 1993 February; 39(2):263-71. Other methods for determining plasma concentration are disclosed in for example Stabler S P, et al, Anal Biochem. 1987 April; 162(1):185-96; Rafii M, et al Anal Biochem. 2007 Dec. 1; 371(1):71-81; Windelberg A, et al; Clin Chem. 2005 November; 51(11):2103-9; Bald E et al; J Chromatogr A. 2004 Apr. 2; 1032(1-2):109-15, Lochman P, et al Electrophoresis. 2003 April; 24(7-8):1200-7 and Ueland P M. Clin Chem. 2008 June; 54(6):1085-6.

This invention further provides novel agents identified by the above-described screening assays and uses thereof for treatments as described herein.

In one aspect of the invention, there is provided a process for preparing a pharmaceutical composition for reducing body fat mass levels comprising:

(a) screening a plurality of compounds by a method which utilises measurement of plasma concentration of a sulphur-containing amino acid e.g. total cysteine concentration, to obtain IC50 values for each compound;
(b) selecting from the plurality a compound having a binding affinity of greater than a predetermined amount, e.g. having an IC50 of less than 500 nM;
(c) synthesising the selected compound; and
(d) incorporating the synthesized compound into a pharmaceutical composition.

Exemplary compounds have an IC50 of less than 1000 nM, more particularly of less than 500 nM, e.g. less than 100 nM, less than 10 nm, less than 1 nM or less than 0.1 nM.

In a further aspect the invention provides a method of identifying a compound capable of reducing body fat mass levels and optionally obesity in a subject comprising assaying the ability of the compound to modulate e.g. reduce plasma total cysteine, thereby identifying a compound capable of reducing obesity in a subject.

In a further aspect the invention provides a method of identifying a compound capable of reducing body fat mass levels in a subject comprising assaying the ability of the compound to modulate e.g. reduce plasma total cysteine concentrations, thereby identifying a compound capable of reducing body fat mass levels in a subject.

Further exemplary details of the invention are described below:

### Example 1

Data was used from 7038 Hordaland Homocysteine Study participants. Multiple linear regression models and dose-response curves were produced to explore the relationship between tCys, tHcy, and BMI. For 5179 participants, associations of tCys and tHcy with fat-mass and lean-mass were investigated. The association of baseline plasma concentrations of plasma cysteine and changes in plasma cysteine with body composition 6 years later was also investigated.

The present invention is based, at least in part, on data from Hordaland Homocysteine Study (HHS-I) and a follow up study, HHS-II (20). HHS-1 was conducted on 18043 residents of the Hordaland county of Western Norway aged 40-42 y (middle-aged) or 65-67 y (elderly). HHS-II was conducted on 7074 subjects. Study protocols for HHS-I and HHS-II have been approved by the Regional Committee for Medical Research Ethics Ethical Committee of Western Norway, whose directives are based on the Helsinki Declaration.

For 7038 subjects, information was available on BMI, plasma tHcy, tCys, and total cysteinylglycine (tCysGly) in both NHS I and II. The cross-sectional association between tHcy, tCys, and BMI was examined using data from HHS-II. The relationship of tHcy and tCys with body total lean-mass and body total fat-mass was examined in 5179 HHS-II participants in whom the fat-mass and lean-mass were measured. The association of changes in tCys and tHcy over the 6-year period with BMI, lean-mass and fat-mass at follow-up was also examined.

For 3516 (including 2894 elderly) of the HHS-II participants, non-fasting plasma concentrations of cystathionine and methionine were measured. Body composition data were available for 2696 of these participants (including 2083 elderly), while BMI data were available for all. Using these data we tested whether the associations of tCys with body composition remained robust after taking into account variations in cystathionine and methionine. Since the measurements of these aminothiols are non-fasting and they are known to vary with food intake (21), analyses involving cystathionine and methionine were adjusted for time since last meal.

### Study Variables

### BMI, Body Composition, and Blood Pressure

Height and weight were measured in light clothing, and BMI was calculated. Seated arterial blood pressure was measured three times for each individual and the average of the second and third measurements is used.

Lean-mass and fat-mass were measured using Dual Energy X-ray Absorptiometry (22), which is based on the different attenuation of photons by different body tissues. Transmission of X-rays at two energy levels allows the derivation of total body bone mineral mass, lean-mass and fat-mass. Measurements were done on one stationary fan beam densitometer [EXPERT-XL, Lunar Corporation, Madison, Wis. (software version 1.72-1.9)]. Coefficient of variation for lean-mass and fat-mass were 1.3% and 1.9%, respectively.

### Lifestyle and Dietary Data

Self-administered questionnaires provided information on diet and lifestyle. Nutrient intakes were calculated using a software system (Kostberegningssystem, version 3.2) developed at the Department of Nutrition, University of Oslo. Physical activity included 2 variables indicating heavy or light physical activity in the past year, with 4 categories within each variable: 1) none, 2)≦1 h/wk, 3) 2-3 h/wk or 4)>4 h/wk. Smoking and coffee consumption were used as continuous variables comprising the number of cigarettes or cups consumed per day.

### Biochemical Measurements

Non-fasting plasma samples were collected in EDTA-containing tubes for tCys, tHcy, tCysGly, folate, and vitamin B12 analyses as previously described (24). Plasma tHcy, tCys and tCysGly were analyzed by HPLC with fluorescence detection. Intra-assay coefficient of variation was lower than 4% (25). LC-MS/MS was used for analyzing methionine, and cystathionine as previously described (26). Plasma folate and vitamin B12 were determined by microbiological assays (27, 28). Serum HDL-cholesterol (HDL) (from HHS II only), triacylglycerol (TG) and total cholesterol were measured using enzymatic methods at the Department of Clinical Chemistry, Ullev\dot{a}l Hospital, Oslo. Creatinine (from HHS II only) was measured in stored plasma using a modification of a liquid chromatography-tandem mass spectrometry described previously (29).

### Statistical Methods

Despite statistically significant interactions between tCys and gender as determinants of BMI and fat-mass (stronger in women than men), and between tHcy and age as determinants of BMI (stronger in younger than in older subjects), stratified analysis showed only modest differences in the patterns and strengths of these associations between middle-aged and elderly men and women. Unless otherwise stated, the four age-gender groups were combined with adjustment for age and gender.

One way ANOVA and Chi square tests with Bonferroni correction were used to determine significant differences between groups, and simple correlations were assessed using Spearman's rank correlation coefficient. Skewed variables were log-transformed prior to analysis. Multiple linear regression models were used to assess the role of tCys as a determinant of BMI, lean-mass and fat-mass. Adjustments were made for variables associated with tCys (8) that are potentially related to body build, and for related metabolites including cystathionine, methionine, and tCysGly.

To reveal non-linear relationships, dose-response curves were constructed to show the estimated difference BMI, lean-mass, and fat-mass by tCys. Gaussian generalized additive regression models were used, as implemented in S-PLUS 6.2 for Windows (Insightful Corporation, Seattle, Wash.). On the y-axis, the model generates a reference value of zero that approximately corresponds to the value of BMI, lean-mass or fat-mass associated with the mean tCys for all subjects. Various models with different co-variates are specified in the figure legends. Corresponding P-values were obtained from multiple linear regression analyses.

To assess the effect of changes in tCys or tHcy over 6 years as predictors of BMI, fat-mass or lean-mass at follow-up, multiple linear regression models were used. BMI, fat-mass or lean-mass at follow-up was used as the dependent variable, whereas changes in tHcy or tCys over 6 years were represented in the models as indicator variables denoting membership to one of the five quintiles for changes in tCys or tHcy. Thus, each regression coefficient estimated the difference in BMI, fat-mass or lean-mass between the lowest quintile and the other four quintiles of changes in tHcy or tCys.

Dose-response curves were also fitted to investigate the effect of increase or decrease in tCys over a 6-year follow-up period on body lean-mass and fat-mass at follow-up. Models took into account the effects of baseline tCys and BMI, as well as changes in plasma lipids and other parameters that may alter body build.

All statistical analyses, except dose-response curves, were performed using the Statistical Package for Social Sciences 12.0 for Windows (SPSS, Chicago. IL). Tests of significance were two-tailed, and P-values <0.05 were considered significant.

### Results

### Characteristics of the Study Population

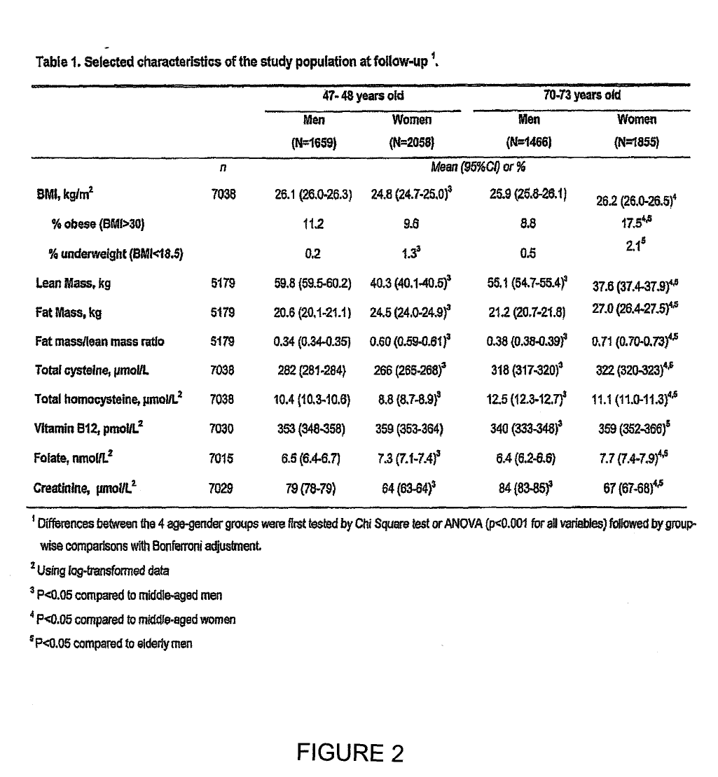

Selected population characteristics are shown in Table 1 (FIG. 2):

BMI was significantly higher in middle-aged men than in women (p<0.001), although men had lower fat-mass than women (p<0.001). The ratio of fat-mass to lean-mass differed significantly (p<0.001) among the 4 age-gender groups, increasing from middle-aged men to elderly men to middle-aged women to elderly women. Using Spearman correlations, lean-mass and fat-mass were positively associated (rₛ=0.28, p<0.001 in men; rₛ=0.37, p<0.001 in women). The correlation of directly measured body weight with the sum of body composition elements as obtained by DXA (lean-mass +fat-mass+bone mineral content) was almost perfect (rₛ=0.98). Mean plasma tCys, tHcy and creatinine were significantly higher in the elderly than in the middle-aged group (p<0.001), and higher in men than women (p<0.05) of the same age group.

tHcy and tCys Interrelationship

Linear regression analysis showed a significant positive association between tHcy as a determinant variable and tCys as dependent variable, adjusting for age and gender (partial r=0.37, p<0.001), which was unchanged by adjustment for folate, vitamin B12, creatinine, fat-mass and lean-mass (data not shown). From low to high tHcy levels, tCys differed by about 60 μmol/L, although towards higher tHcy values, tCys concentrations leveled off. Change in tHcy over 6 years was linearly associated with change in tCys in the same direction (partial r=0.32, p<0.001), with adjustment for age and gender. Because of this strong interrelationship between tHcy and tCys, the linear regression analyses were always performed first with one variable then with both variables included.

tCys and Indices of Body Mass
tCys and BMI

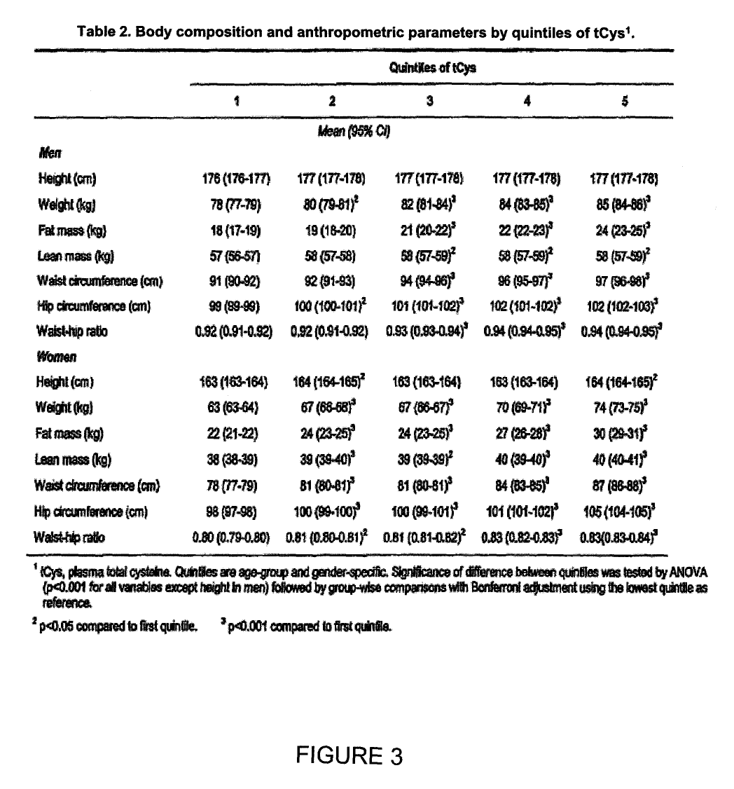

The association between tCys and BMI, controlling for age and gender, was linear, highly significant (partial r=0.28, p<0.001; FIG. 3-A), and not affected by adjustments for tHcy (partial r for tCys=0.29, p<0.001), or plasma creatinine, lipids (total cholesterol, TG, HDL), coffee intake and systolic blood pressure (partial r for tCys=0.26, p<0.001). In a model fully adjusted for age, gender, tHcy, creatinine, lifestyle factors (coffee intake, smoking, physical activity), nutritional intake (total energy, protein and fat intakes) and serum lipids, tCys was the strongest determinant of BMI (partial r=0.23, p<0.001).

tCys and Body Composition

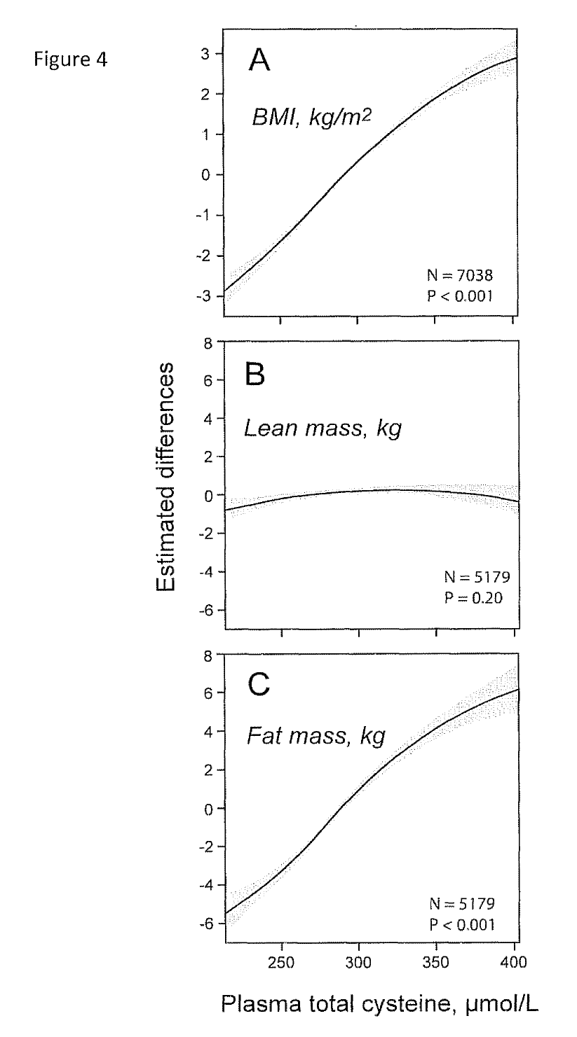

There was no effect of tCys on lean-mass when fat-mass was taken into account, and the dose response curves were essentially horizontal with (partial r=0.02, p=0.10) and without (r=0.02, p=0.20; FIG. 4-B) adjustment for tHcy. There was a strong positive linear association between tCys and fat-mass, after controlling for age, gender and lean-mass (partial r=0.26, p<0.001; FIG. 4-C). This association was largely unaffected by adjustment for tHcy, plasma creatinine, total cholesterol, TG, HDL, coffee intake and systolic blood pressure (partial r for tCys=0.25, p<0.001). In this latter model, tCys was the strongest plasma determinant of fat-mass, followed by HDL (partial r=−0.21, p<0.001) and TG (partial r=0.1, p<0.001). In a fully adjusted model, including age, gender, lean-mass, lifestyle (coffee consumption, smoking, physical activity), nutritional (total energy, protein and, fat intakes) and plasma variables (creatinine, lipids and tHcy), tCys was second only to gender and lean-mass as determinant of fat-mass (partial r for tCys=0.21, p<0.001).

Lean-mass, fat-mass and anthropometric measures by quintiles of tCys in men and women are shown in Table 2 (FIG. 3). Women in the highest quintile of tCys had an average weight, fat-mass and waist circumference that were respectively 11 kg, 9 kg and 9 cm higher compared to those in the lowest quintile. For men, the difference between the highest and lowest tCys quintiles was slightly less pronounced (7 kg, 6 kg and 6 cm, respectively; p<0.001 for ANOVA across quintiles in men and women). Waist and hip circumferences and waist/hip ratio also increased significantly across tCys quintiles. In contrast, height showed only a minor fluctuation of up to 1 cm between the tCys quintiles with no apparent trend.

Change in tCys over a 6-year follow-up period was associated with significant differences in BMI and fat-mass, with a negligible effect on lean-mass. Estimated difference in BMI, fat-mass and lean-mass at follow-up by quintiles of change in tCys compared to first quintile, adjusting for various covariates, is shown in Table 3 (FIG. 5). The group of participants with the highest increase in tCys had a fat-mass at follow-up that was >2 kg higher than the reference category (p for trend <0.001), with adjustment for baseline tCys.

Dose response curves showed that a 10% reduction in tCys was associated with a fat mass at follow-up that was ˜2 kg lower than that associated with no change in tCys, with adjustment for age, gender, lean-mass, baseline BMI and baseline tCys (partial r for change in tCys=0.12, p<0.001, FIG. 6-A). The same effect was observed with additional adjustment for changes in plasma lipids and smoking habits, as well as physical activity, blood pressure and creatinine at follow-up (partial r for change in tCys=0.12, p<0.001, FIG. 6-C).

### Other Plasma Sulfur Amino Acids

It was investigated whether the plasma concentrations of other aminothiols involved in the cysteine metabolic pathway could explain the strong association of tCys with fat-mass. A multiple linear regression model examined the roles of tCys, tHcy, cystathionine, methionine and tCysGly as predictors of fat-mass after adjustment for age, gender, lean mass, and time since last meal. tCys remained the strongest aminothiol determinant (partial r=0.25, p<0.001), followed by cystathionine (partial r=0.1, p<0.001). Methionine and tHcy showed weak inverse associations with fat-mass (partial r=−0.04, p=0.038, and partial r=−0.05, p=0.011 respectively), while tCysGly showed no significant association (partial r=0.03, p=0.161). With further adjustment for plasma lipids, only tCys (partial r=0.24, p<0.001), and cystathionine (partial r=0.05, p=0.011) and tHcy (partial r=−0.06, p=0.002) remained significantly associated with fat-mass.

Previous studies have indicated that plasma total cysteine (tCys) is related to BMI in the Hordaland Homocysteine Study (HHS) and has interpreted this relation as change in tCys as a result of change in BMI. The inventors have considered for the first time that the converse is true; that BMI is directly affected by changes in tCys. Thus, excess body fat levels in a subject may be controlled by controlling levels of tCys. This indication gives rise to potential new treatments for obesity and other body mass related disorders.

### Discussion

tCys and Indices of Body Mass

In the present study, tCys showed no association with lean-mass, but a strong positive association with fat-mass. As a determinant of fat-mass, tCys was stronger than even serum lipids such as TG, HDL and total cholesterol. The relationship between cysteine and fat mass was completely independent of dietary and lifestyle factors, including physical exercise, protein, fat and total energy intakes, as well as smoking and coffee consumption. The examples suggest that high tCys or a related factor is causally related to body fat and obesity in the general population.

### Example 2

To exclude the possibility that the Hordaland study findings are restricted to the Norwegian population studied, the relation of plasma cysteine with body weight in 1550 subjects from 9 European countries (COMAC cohort) were also examined. A strong positive association of plasma cysteine with body weight and BMI was observed. Moreover, the inventors report a marked increase of odds of obesity associated independently with increasing tCys.

### Subjects and Methods

### Study Population

The COMAC study population comprises a total of 750 vascular disease cases (544 men, 206 women) and 800 controls (570 men, 230 women), all under 60 years of age. Cases and controls were recruited from 19 centers in 9 European countries. Details on subject recruitment and data collection have been published previously (19). Selected characteristics of the population are shown in FIG. 8 (Table 4).

Cases had defined clinical and investigational evidence of coronary, cerebrovascular or peripheral vascular disease, and were mostly recruited within one year of diagnosis, to avoid a possible influence of treatment on the variables studied (19). Controls were free of overt disease, and 50% of these subjects were from community samples, 33% were from employee health insurance registers, and 17% were hospital employees. Two percent of control subjects were hospital patients. Exclusion criteria included non-atherosclerotic vascular disease, cardiomyopathy, diabetes mellitus, pregnancy, psychiatric illness, renal or thyroid disease, anticonvulsant therapy, and recent (<3 months) systemic illness or exposure to nitrous oxide.

### Study Variables

Data collected included information about age, sex, smoking habits, blood pressure, weight, height, and drug and vitamin usage, as well as biochemical measurements. BMI was calculated as body weight divided by the square of height in meters. For both systolic and diastolic blood pressure, the mean of 4 values was used.

### Plasma Variables

Fasting and post-methionine load plasma tCys, total homocysteine (tHcy) and total cysteinylglycine (tCysGly) were measured by high-performance liquid chromatography (HPLC) with fluorescence detection (20). Measurements of serum lipids, folate, vitamin B12, and creatinine were performed at Mime-AB, as described (19). Fasting plasma concentrations of the aminothiols were used in the present study.

### Statistical Methods

Positively skewed variables, namely GGT (gamma glutamyltransferase), tHcy, creatinine and triacyl glycerol were log-transformed prior to analysis.

### Linear Regression

Multiple linear regression models as implemented in Statistical Package for Social Sciences 12.0 for Windows (SPSS, Chicago. IL) were used to assess the following associations:
1—GGT as a determinant of tCys, with adjustment for various covariates, including known tCys determinants.
2—GGT as a predictor of BMI, with and without adjustment for tCys.
3—tCys as a determinant of BMI, adjusting for potential confounders, including GGT.

### Logistic Regression

Using multivariate-adjusted logistic regression we calculated the odds ratio for obesity (defined as BMI ≧30 kg/m2) per quartile of tCys with and without adjustment for GGT and other confounders.

All models were adjusted for age, gender, and diagnosis (case vs. control). Choice of other potential confounders was based on preliminary simple Spearman correlations and known biological associations. Depending on the model, adjustments were made for blood pressure, smoking habits, plasma concentrations of tHcy, creatinine, lipids, tCysGly, urea and serum glutamic-oxalacetic transferase (SGOT).

### Dose-Response Curves

To reveal non-linear relationships, dose-response curves were constructed to show the associations described above, namely GGT with tCys, GGT with BMI and tCys with BMI. Gaussian generalized additive regression models, as implemented in S-PLUS 6.2 for Windows (Insightful Corporation, Seattle, Wash.) were used. On the y-axis, the model generates a reference value of zero that approximately corresponds to the value of dependent variable associated with the mean of the independent variable (tCys) for all subjects. Various models with different sets of covariates are specified in the figure legends. Corresponding P-values and partial correlation coefficients were obtained from multiple linear regression analyses.

### Results

### Population Characteristics

Selected characteristics of the study population according to case-control status and gender are shown in Table 4 (FIG. 8).

GGT as a Predictor of tCys and tCysGly

In a multiple linear regression model adjusted for age, gender, diagnosis, and other tCys determinants including tHcy and creatinine, GGT as an independent variable showed a modest positive linear association with tCys as dependent (partial r=0.05, p=0.049; FIG. 9). Using a similar model to examine the role of GGT as a predictor of tCysGly, the positive association of GGT with tCysGly was statistically non-significant (partial r=0.05, p=0.087).

tCys as a Predictor of BMI

A significant positive association was observed between tCys and BMI, with adjustment for age, gender, and case-control status (partial r=0.19, p<0.001). The association was linear, resulting in a difference in BMI of about 3 kg/m² from the second to 99^{th} tCys percentile (FIG. 10-A). The association remained robust with sequential addition of plasma GGT, tCysGly, creatinine and lipids (triacyl glycerol, HDL and LDL cholesterol) to the model (partial r for tCys=0.19, p<0.001). In a model adjusted for age, gender, case-control status, blood pressure and smoking habits, as well as plasma concentrations of GGT, tCysGly, tHcy, lipids, creatinine, urea and SGOT, tCys remained the single strongest independent predictor of BMI (partial r=0.19, p<0.001; FIG. 10-B), followed by HDL cholesterol (partial r=−0.16, p<0.001). In a subset of 818 subjects where plasma taurine and glutathione measurements were available, adjusting for these downstream products of cysteine had no effect on the association of tCys with BMI.

### Plasma GGT as a Predictor of BMI

A significant positive association was observed by linear regression between plasma GGT and BMI, with adjustment for age, gender, and case-control status (partial r=0.17, p<0.001; FIG. 10-C). The association of GGT with BMI was unaffected by adjustment for tCys (partial r=0.17, p<0.001), but was substantially weakened by addition of other confounders to the model (smoking habits, blood pressure, plasma concentrations of triacyl glycerol, HDL and LDL cholesterol, tCysGly, tHcy, creatinine, urea and SGOT; partial r=0.07, p=0.016; FIG. 10-D).

tCys and Risk of Obesity

FIG. 11 (table 5) shows the odds ratio (OR) of obesity, defined as BMI >30 kg/m², for each quartile of tCys, compared to the lowest quartile. tCys was significantly and independently associated with risk of obesity in the crude and multivariate adjusted models. Even after adjustment for blood pressure, smoking habits and plasma/serum concentrations of TG, tHcy, creatinine and SGOT, the odds ratio for obesity in the highest vs. lowest tCys quartile was 3.5 (95% CI: 1.8-6.8, p<0.001). Further adjustment for tCysGly, urea, and HDL-C and LDL-C had a negligible effect on these results (data not shown).

Thus, the odds of being obese were 3.5 times higher in the 25% of the population with the highest tCys, compared to those with the lowest tCys, after taking into account various lipid-related and lifestyle factors.

### Discussion

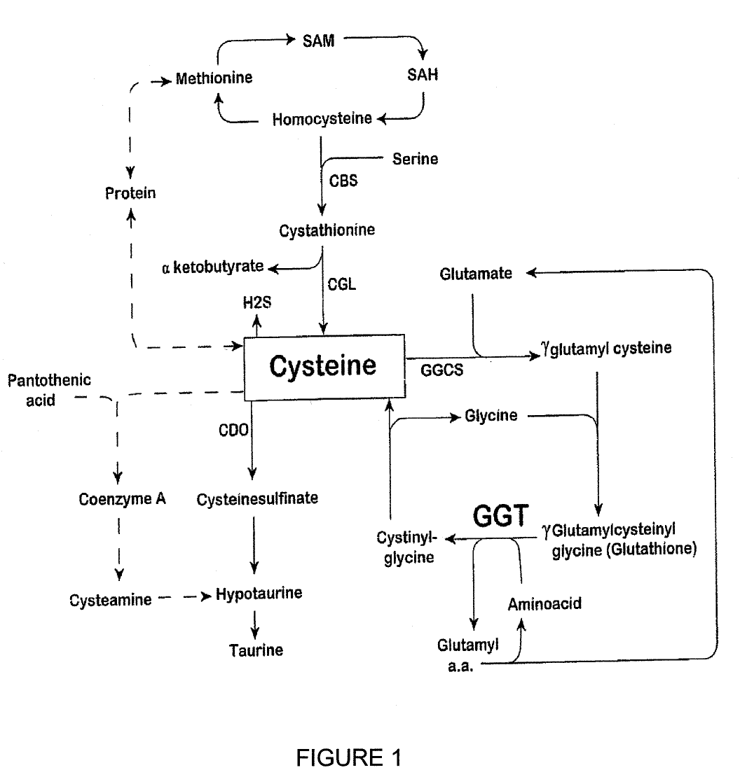

The associations of tCys and GGT with BMI were studied in 1550 subjects recruited from 19 centers in 9 European countries. The study aimed to confirm the association reported under example 1 and to determine whether GGT is the underlying mechanism linking tCys to BMI (for the metabolic relation of GGT with tCys see FIG. 1). The data confirm the results of Example 1 and provide evidence that the tCys-BMI association is independent of GGT.

Plasma GGT was positively associated with tCys, after controlling for the major cysteine determinants, age, gender, homocysteine and creatinine (as a renal function marker). tCys was strongly associated with BMI, independent of and stronger than GGT, cysteinylglycine, plasma lipids, and renal and liver function markers. In fact in all models in which tCys was plotted as predictor variable, tCys was the single strongest determinant of BMI. Moreover, subjects in the upper tCys quartile were 3.5 times as likely to be obese, compared to those in the lowest quartile, after adjustment for metabolic and lifestyle confounders. The results also show that the association of tCys with BMI cannot be explained by their mutual association with GGT.

It therefore follows that cysteine or one of its downstream products could play a causal role in regulating body weight.

### Example 3

A link between two disorders which have defect in the cystathionine beta-synthase (CBS) gene, which encodes for the enzyme responsible for cysteine synthesis, and body fat has been considered. Firstly, a disorder known as CBS deficiency is characterised by marked reduction in cysteine. CBS deficiency not only leads to upstream accumulation of homocysteine and methionine, but also to reduced synthesis of cystathionine and cysteine. Sufferers of this disorder have a thin phenotype with low BMI, decreased subcutaneous fat and body weight frequently below the 5^{th} percentile. The inventors have considered, for the first time, the link between the cysteine levels in CBS sufferers and body fat levels.

In addition, subjects with Down's syndrome overexpress the CBS gene by up to 50% due to the localisation of the CBS gene on the “tripled” chromosome 21. Individuals with Down's syndrome have a higher prevalence of ‘1 obesity. The inventors have considered for the first time that the higher prevalence of obesity in Down's syndrome sufferers is at least in part due to increased cysteine levels as a result of overexpression of the CBS gene. The present invention includes a link between cysteine levels and body fat levels and thus treatments for excessive body fat levels in a patient.

### Example 4

As shown above in example 1, the Hordaland Study indicates that the powerful association of cysteine with body fat is not shared by other sulfur amino acids.

An experiment was conducted in rats to investigate whether body weight and visceral fat could be decreased by dietary reduction of tCys. Since cysteine is readily synthesized in humans and rodents from methionine via the transsulfuration pathway, plasma cysteine was decreased by restricting intake of its precursor, methionine. An experimental (tCys-lowering) diet was used which was devoid of cysteine and also very low in its methionine content. The experiment described below investigates whether body weight and visceral fat can be decreased by dietary reduction of plasma cysteine availability in the rat.

### Materials and Methods

Four-week-old male Fischer-344 rats (N=22) were maintained one rat per cage on a 12 h light/dark cycle and fed a standard control diet for 2 weeks. At 6 weeks of age, the rats were randomly assigned to control or tCys-lowering diets and maintained on these diets for 12 weeks. The tCys-lowering diet was deficient in methionine (1.7 g/kg in the tCys-lowering diet versus 8.6 g/kg in control diet; see Table 6), to avoid possible endogenous cysteine synthesis from methionine. The sulfur amino acid reduction in the tCys-lowering diet was compensated by raising the glutamic acid content on an equal gram basis. Food and water were provided ad libitum to both groups throughout the experiment.

TABLE 6
Composition of the tCys-lowering diet: devoid of cysteine, low in
methionine.

| Ingredient | Amount, g/kg | Ingredient | Amount, g/kg |
|---|---|---|---|
| L-Methionine | 1.7 | L-Phenylalanine | 11.6 |
| Glutamic acid | 27.0 | Glycine | 23.3 |
| L-Arginine | 11.2 | Corn oil | 80.0 |
| L-Lysine | 14.4 | Dextrin | 50.0 |
| L-Histidine | 3.3 | Cornstarch | 436.1 |
| L-Leucine | 11.1 | Sucrose | 200.0 |
| L-Isoleucine | 8.2 | Solka-floc | 50.0 |
| L-Valine | 8.2 | Choline bitartrate | 2.0 |
| L-Tryptophan | 1.8 | Vitamin mix | 10.0 |
| L-Threonine | 8.2 | Mineral mix | 35.0 |

After completing the dietary regimen, the rats were weighed, and then anesthetized using an Euthenex Easy Anesthesia system (Palmar, Pa.). Blood was collected from the subclavian vein for biochemical analysis. Visceral fat mass was determined by surgical excision and weighing of the inguinal, epididymal and retroperitoneal fat pads.

### Assay Methods

Commercially available rat ELISA kits were used to measure leptin (Assay Design) and IGF-1 (IDS). Insulin and adiponectin were measured by RIA (Linco Research, Inc., St. Charles, Mo., USA). Triglycerides, cholesterol, and glucose were determined using a Beckman Synchron CX5 clinical system (La Brea, Calif.).

LC-MS/MS was used for analyzing tCys and other sulfur aminoacid concentrations using modification of a method previously described (Refsum H, Grindflek A W, Ueland P M, et al. Clin Chem 2004; 50:1769-84).

### Results

Table 7 below shows the distributions (median, 25^{th} and 75^{th} percentiles) of the plasma concentrations of sulfur aminoacids, lipids and adipokines, as well as body weight parameters in experimental and control rats after three months on a tCys lowering diet.

TABLE 7

| | | | | | Exp/Control |
|---|---|---|---|---|---|
| | Control | | Experimental | | (%) |
| Sulfur aminoacids and tGSH² | | | | | |
| Methionine, μmol/L | 91 | (81-124)³ | 35 | (34-38) | 38 |
| tHcy, μmol/L | 18.2 | (16.0-20.8) | 46.1 | (30.3-53.4) | 253 |
| Cystathionine, μmol/L | 2.40 | (2.09-2.47) | 1.38 | (1.29-1.61) | 58 |
| tCys, μmol/L | 250 | (238-268) | 139 | (136-155) | 56 |
| Taurine, μmol/L | 280 | (277-349) | 82 | (52-97) | 29 |
| tGSH, μmol/L | 26.1 | (24.1-28.9) | 21.7 | (19.4-24.3) | 83 |
| Body weight and metabolic | | | | | |
| parameters | | | | | |
| Body weight, g⁴ | 338 | (315-385) | 188 | (188-184) | 56 |
| Visceral fat, g | 22 | (17-24) | 9 | (9-10) | 41 |
| Visceral fat/body | 6.1 | (5.2-7.0) | 4.9 | (4.4-5.4) | 80 |
| weight % | | | | | |
| Leptin, pg/ml | 10696 | (8311-13558) | 2315 | (1497-3132) | 22 |
| Adiponectin, μg/ml⁴ | 3.9 | (3.5-6.9) | 12.8 | (12.1-13.7) | 329 |
| IGF-1, ng/ml⁴ | 1340 | (1240-1440) | 568 | (500-600) | 42 |
| Insulin, ng/ml⁴ | 1.26 | (0.90-1.34) | 0.50 | (0.28-0.56) | 40 |
| Glucose, mg/dl⁴ | 186 | (170-203) | 161 | (155-171) | 87 |
| Triglycerides, mg/dl⁴ | 96 | (85-128) | 32 | (26-41) | 33 |
| Cholesterol, mg/dl⁴ | 47 | (45-48) | 38 | (34-41) | 81 |

(1) Data presented as median (25^{th}-75^{th} %). N = 11 rats per group.
²tGSH, total glutathione; tHcy, total homocysteine; tCys, total cysteine.
³All parameters significantly different in experimental rats versus control by Mann-Whitney U test. P < 0.001 for all except tGSH, visceral fat/body weight % and glucose: P ≦ 0.007.

Notably, median tCys and median body weight were each reduced to 56% of control (p<0.001). Plasma methionine, cystathionine and taurine were also significantly decreased. Plasma total glutathione was modestly but also significantly lower (p=0.004). In contrast, tHcy was markedly elevated in rats fed the tCys-lowering diet, averaging more than double control values (p<0.001).

Body weight, visceral fat, and the ratio of visceral fat to total body weight were significantly (p<0.001) lower in experimental rats at the end of 3 months (FIG. 12). Experimental rats also exhibited a favourable lowering of plasma concentrations of leptin, insulin, IGF-1, glucose, triglycerides and total cholesterol, and elevation of adiponectin (p≦0.007 for all; Table 7).

This experiment was conducted to investigate whether the strong association between tCys and fat mass in humans is due to an effect of cysteine on fat mass rather than an influence of fat mass on tCys. Assigning rats to a tCys-lowering diet for 3 months lowered their tCys by 44% relative to control and achieved a significant reduction of their body weight (by 44%) and visceral fat mass (by 59%) compared to control. Other protective effects related to the body weight reduction in the experimental rats included a lowering of plasma glucose, insulin and lipids, and elevation of adiponectin.

This experiment provides evidence for the causal relationship between cysteine and fat mass and demonstrates that diets reducing tCys readily decrease weight gain and visceral fat. Thus decreasing tCys in humans by dietary or pharmaceutical approaches may be an effective intervention for treatment of obesity.

In this regard, anti-cysteine compounds may be first tested in suitable animal models, including transgenic mice models developed particularly in relation to obesity. Suitable animal models include those disclosed in Speakman et al (Obes Rev. 2007 March; 8 Suppl 1:55-61.)

To distinguish the effect of cysteine on body weight from that of methionine, the same experiment described above was repeated with addition of a third group. In addition to the methionine restricted (MR) and control groups, a third group was included comprising rats fed an MR diet supplemented with L-cysteine at a concentration of 5 g/kg diet. Age of the rats at the start of experiment, housing conditions and MR and control diets were similar to those in the above experiment. Food and water were provided ad libitum. Each group consisted of 8 rats; there was no significant difference in body weight between the 3 groups at baseline. Preliminary results after 3 weeks into the study are shown in the table below.

TABLE 8
Body weights and food consumption after 3 weeks¹.

| | Control | MR | MR + Cys |
|---|---|---|---|
| Body weight, g | 197 ± 22 | 160 ± 14 | 199 ± 15² |
| Food intake, g/d | 17 ± 1 | 20 ± 4 | 16 ± .3 |

¹Data presented as mean ± SD. N = 8 rats per group. MR, methionine-restricted; MR + Cys, methionine-restricted supplemented with 0.5% L-cysteine.
²Significantly different from the MR group at p < 0.001.

These preliminary data show that addition of L-cysteine to a MR diet prevented the decrease in weight gain observed in MR rats. Given that cysteine cannot be converted to methionine in the body (the CBS reaction initiating cysteine synthesis from methionine is irreversible), this demonstrates a direct effect of cysteine on body weight. Assessments of visceral fat and plasma sulfur amino acid concentrations will be made at the end of the study (total study duration=12 weeks).

This experiment provides evidence for the causal relationship between cysteine and fat mass and demonstrates that diets reducing tCys readily decrease weight gain and visceral fat, and that adding cysteine to these diets prevents the decrease in weight gain. Thus decreasing tCys in humans by dietary or pharmaceutical approaches may be an effective intervention for treatment of obesity.

### Example 5

To further confirm the association of cysteine reduction with decreased fat mass, the effect of block of cysteine action on adipose tissue is investigated in rodents. Specifically, the experiment investigates the effect of the drug sulfasalazine, which blocks cellular uptake of cysteine by inhibiting the cysteine transporter on body weight and fat mass of mice. The action of sulfasalazine will be tested in relation to dietary- or genetically-induced obesity, as well as “normal” body weight.

### Materials and Methods

60 adult male mice belonging to one of 3 body-weight groups as outlined below are initially maintained on a standard rat pellet diet and water ad libitum under controlled light-dark cycles (7 a. m. to 7p.m.), humidity, and temperature (20-22° C.) conditions.

After acclimatization, rats are randomized to either sulfasalazine 500 mg/kg body weight in corn oil vehicle or placebo (corn oil vehicle only) for 40 days.

Dietary obesity is induced prior to start of the experiment by a palatable high-fat diet as previously described (Speakman J et al Obes Rev. 2007 March; 8 Suppl 1:55-61). From the start of the experiment, high fat diet will be stopped, and all 60 mice will have free access to standard laboratory diet and water.

The results will be measured in terms of total body weight and total fat mass, as assessed by dual-energy X-ray absorptiometry. Body weight will be measured weekly. Body fat mass and all the outcome variables listed below will be assessed on the first day of drug treatment and then every 20 days thereafter for 60 days.

TABLE 9

Plasma concentrations of sulfur amino
acids
Methionine
Total homocysteine
Cystathionine
Cysteine
Taurine
Plasma lipid and metabolic profile
Triglycerides
Total cholesterol
Free fatty acids
Glucose
Oral glucose tolerance
Body fat mass using dual-energy X-ray
Absorptiometry

Block of cysteine uptake by cells by sulfasalazine should lead to cysteine starvation (Doxsee, D. W., et al. Prostate 2007; 67 (2), pp. 162-171) preventing any potential adipogenic action of cysteine on adipose tissue cells, which should result in weight loss (or decreased weight gain) in mice treated with sulfasalazine versus control. The results of the experiment will indicate which group of mice (genetically obese, dietary obese or normal weight) shows the most favourable response in terms of fat mass reduction.

Example 5 can be carried out to determine the effect of other anti-cysteine agents on body fat mass, such as cilastatin, which is an inhibitor of dipeptidases that release cysteine from cysteinylglycine and acetaminophen which is a stimulator of cysteine turnover and acts to stimulate the enzyme cysteine dioxygenase or the enzyme gamma glutamyl cysteine synthetase.

### Example 6

This example describes the ability of baseline plasma cysteine concentrations to predict increase in fat mass 6 years later. The most important predictor of what an individual's body weight will be 6 years later is the same individual's body weight at baseline. However, even after taking into account the baseline BMI of the study participants, the inventors show that baseline cysteine levels affect fat mass measured 6 years later.

This analysis is based on data from 7054 middle-aged and elderly men and women recruited from the general population in the Hordaland Homocysteine Study. Body mass index and plasma concentrations of cysteine, homocysteine, cholesterol and triglycerides were measured in all subjects at 2 time points 6 years apart. Throughout this example, the first assessment will be referred to as “baseline” and the second assessment as “follow-up”.

Subjects at baseline were divided into 4 roughly equal groups according to their baseline plasma cysteine concentrations (cysteine quartiles), taking into account their age and gender. These subjects were then followed up and their fat mass determined 6 years later. The mean fat mass of subjects in the 4 quartiles of baseline plasma cysteine was calculated, taking into account their baseline BMI, as well as their baseline plasma triglycerides, cholesterol and homocysteine.

Compared to the first quartile (quarter of the population having the lowest cysteine concentrations), those in the 4^{th} quartile (highest plasma cysteine concentrations) had an average of 1 kg higher fat mass at follow-up, independent of their initial BMI and plasma lipids. The difference was highly significant (p<0.0001). This is analogous to reporting that if all study subjects started with the same body weight and plasma lipids at baseline, the 25% of the study population with the highest cysteine levels will be 1 kg fatter at follow-up. This one kg is purely fat, as measured by dual-energy x-ray absorptiometry, one of the most accurate methods to date of measuring body fat.

### Example 7

### Exclusion of Reverse Causality

### Bariatric Surgery Patient Population

It was investigated whether tCys is associated with obesity due to increased fat mass increasing tCys. Sixty morbidly obese individuals were investigated before and after weight-loss surgery and it was established that fat mass does not determine plasma tCys, thus indicating that elevated tCys promotes obesity rather than the other way round. The study is described in detail below:

### Subjects:

Sixty extremely obese subjects with BMI >40 kg/m² (mean 55±3 kg/m²) were studied. The subjects underwent one of two types of weight loss procedures: gastric bypass or duodenal switch. Both surgeries are followed by severely reduced food intake and hence rapid and profound loss of body fat. A 6-month follow-up was completed. Sixty normal weight controls were used.

### Methods

Weight, height and tCys were measured immediately before (baseline) and 6 weeks and 6 months after the weight-loss surgery (follow-up). The same parameters were also estimates in the sixty normal weight control subjects at baseline. tCys was assayed by an established Liquid Chromatography Tandem-Mass Spectrometry method. BMI was calculated as weight (kg)/height (m)². Statistical analysis was performed using the Statistical Package for Social Sciences 12.0 for Windows (SPSS, Chicago, Ill.). Paired samples t-test was used to compare measurements pre- and post-surgergy. P<0.05 was considered significant.

### Results:

Table 10 below shows tCys, BMI and body weight measurements in the control group as well as in the morbid obese group pre- and post-surgery. Together with a dramatic decrease in BMI of ˜15 kg/m² (equivalent to average weight loss of 45 kg; p<0.001 for weight and BMI), the change in tCys was non-significant (see FIG. 13). These findings establish that cysteine is not produced or released to the plasma in proportion to body fat, i.e. it excludes the possibility that fat mass is the causal determinant of tCys.

TABLE 10
Plasma tCys and body weight parameters pre- and post-surgery¹

| | Control | | | | P value | P value |
|---|---|---|---|---|---|---|
| | Group | Obese Group (N = 60) | | | Preop. | Preop. |
| | (N = 60) | Preop | 6 wk | 6 mo | vs. 6 wk | vs. 6 mo |
| BMI, | 23.7 ± 2.9 | 55.0 ± 3.3 | 47.5 ± 3.2 | 40.0 ± 4.0 | <0.001 | <0.001 |
| kg/m² | | | | | | |
| Weight, | 72 ± 11 | 162 ± 22 | 140 ± 20 | 118 ± 19 | <0.001 | <0.001 |
| kg | | | | | | |
| tCys, | 268 ± 32 | 308 ± 43 | 309 ± 42 | 301 ± 50 | 0.93 | 0.22 |
| μmol/L | | | | | | |

¹Data presented as mean ± SD. Groups were compared by paired-samples T test. P < 0.05 was considered significant.

### REFERENCES

1. Mudd S H, Levy H L, Skovby F. The Molecular and Metabolic Bases of Inherited Disease. 7th ed. New York McGraw-Hill, 1995:1279-1327.
2. Brosnan J T, Brosnan M E. J Nutr 2006; 136:1636 S-1640S.
3. Hargreaves I P, Lee P J, Briddon A. Amino Acids 2002; 22:109-18.
4. Brenton D P, Dow C J, James J I, Hay R L, Wynne-Davies R. J Bone Joint Surg Br 1972; 54:277-98.
5. Gibson J B, Carson N A, Neill D W. J Clin Pathol 1964; 17:427-37.
6. Herrmann M, Widmann T, Herrmann W. Bone 2006; 39:1385-6; author reply 1386-7.
7. Kraus J P, Ko\check{z}ich V. Cystathionine β synthase and its deficiency. In: Carmel R, Jacobsen D W, eds. Homocysteine in Health and Disease. First ed. Cambridge: Cambridge University Press, 2001:223-244.
8. El-Khairy L, Ueland P M, Nygard O, Refsum H, Vollset S E. Lifestyle and cardiovascular disease risk factors as determinants of total cysteine in plasma: the Hordaland Homocysteine Study. Am J Clin Nutr 1999; 70:1016-24.
9. El-Khairy L, Vollset S E, Refsum H, Ueland P M. Clin Chem 2003; 49:113-20.
10. Jacques P F, Bostom A G, Wilson P W, Rich S, Rosenberg I H, Selhub J. Am. J Clin Nutr 2001; 73:613-21.
11. Koehler K M, Romero L J, Stauber P M, et al. J Am Coll Nutr 1996; 15:364-76.
12. Poirier L A, Wise C K, Delongchamp R R, Sinha R. Cancer Epidemiol Biomarkers Prey 2001; 10:649-55.
13. Brasileiro R S, Escrivao M A, Taddei J A, D'Almeida V, Ancona-Lopez F, Carvalhaes J T. Nutr Hosp 2005; 20:313-9.
14. Dixon J B, Dixon M E, O'Brien P E. Int J Obes Relat Metab Disord 2001; 25:219-27.
15. Henning B F, Tepel M, Riezler R, Gillessen A, Doberauer C. Res Exp Med (Berl) 1998; 198:37-42.
16. Sheu W H, Wu H S, Wang C W, Wan C J, Lee W J. Intern Med 2001; 40:584-8.
17. Rauh M, Verwied S, Knerr I, Dorr H G, Sonnichsen A, Koletzko B. Amino Acids 2001; 20:409-18.
18. Gallistl S, Sudi K M, Erwa W, Aigner R, Borkenstein M. Metabolism 2001; 50:1220-3.
19. Gallistl S, Sudi K, Mangge H, Erwa W, Borkenstein M. Diabetes Care 2000; 23:1348-52.
20. Refsum H, Nurk E, Smith A D, et al. J Nutr 2006; 136:1731 S-1740S.
21. Guttormsen A B, Solheim E, Refsum H. Am J Clin Nutr 2004; 79:76-9.
22. Pietrobelli A, Formica C, Wang Z, Heymsfield S B. D Am J Physiol 1996; 271:E941-51.
23. Nes M, Frost Andersen L, Solvoll K, et al. Eur J Clin Nutr 1992; 46:809-21.
24. Nygard O, Vollset S E, Refsum H, et al. The Hordaland Homocysteine Study. Jama 1995; 274:1526-33.
25. Fiskerstrand T, Refsum H, Kvalheim G, Ueland P M. Clin Chem 1993; 39:263-71.
26. Refsum H, Grindflek A W, Ueland P M, et al. Clin Chem 2004; 50:1769-84.
27. Molloy A M, Scott J M. Methods Enzymol 1997; 281:43-53.
28. Kelleher B P, Broin S D. J Clin Pathol 1991; 44:592-5.
29. Holm P I, Ueland P M, Kvalheim G, Lien E A. Clin Chem 2003; 49:286-94.
30. Leuchtenberger C, Leuchtenberger R. Cell Biol Int Rep 1977; 1:317-24.
31. Pieniazek D, Rakowska M, Szkilladziowa W, Grabarek Z. Br J Nutr 1975; 34:175-90.
32. Reid I R, Ames R, Evans M C, et al. J Clin Endocrinol Metab 1992; 75:45-51.
33. Namekata K, Enokido Y, Ishii I, Nagai Y, Harada T, Kimura H. J Biol Chem 2004; 279:52961-9.
34. Moat S J, Bonham J R, Allen J C, Powers H J, McDowell I F. J Inherit Metab Dis 1999; 22:243-6.
35. Tsuboyama-Kasaoka N, Shozawa C, Sano K, et al. Endocrinology 2006; 147:3276-84.
36. Whitfield J B. Crit. Rev Clin Lab Sci 2001; 38:263-355.
37. Achouri Y, Robbi M, Van Schaftingen E. Biochem J 1999; 344 Pt 1:15-21.
38. Simpson R C, Hill F W, Freedland R A. J Nutr 1975; 105:379-84.
39. Stipanuk M H. Annu Rev Nutr 2004; 24:539-77.
40. Chua B H, Giger K E, Kleinhans B J, Robishaw J D, Morgan H E. Am J Physiol 1984; 247:C99-106.
41. Lopaschuk G D, Hansen C A, Neely J R. Am J Physiol 1986; 250:H351-9.
42. Cupo M A, Donaldson W E. Nutrition Reports International 1986; 33:147-155
43. Guyton A C, Hall J E. Textbook of Medical Physiology. 11 ed. Philadelphia.: Elsevier Saunders, 2006.
44. Large V, Peroni O, Letexier D, Ray H, Beylot M. Diabetes Metab 2004; 30:294-309.
45. Fulghesu A M, Ciampelli M, Muzj G, et al. Fertil Steril 2002; 77:1128-35.
46. Blouet C, Mariotti F, Azzout-Marniche D, et al. Free Radic Biol Med 2007; 42:1089-97.
47. Ganji V, Kafai M R. Demographic, health, lifestyle, and blood vitamin determinants of serum total homocysteine concentrations in the third National Health and Nutrition Examination Survey, 1988-1994. Am J Clin Nutr 2003; 77:826-33.
48. Battezzati A, Bertoli S, San Romerio A, Testolin G. Nutr Metab Cardiovasc Dis 2007; 17:525-34.
49. McCarty M F. Med Hypotheses 2001; 56:5-7.
50. Kuo H K, Liao K C, Leveille S G, et al. J Gerontol A Biol Sci Med Sci 2007; 62:434-9.
51. Kado D M, Bucur A, Selhub J, Rowe J W, Seeman T. Am J Med 2002; 113:537-42.
52. McDermott M M, Ferrucci L, Guralnik J M, et al. J Am Coll Cardiol 2007; 50:897-905.
53. Hurwitz L J, Chopra J S, Carson N A. Acta Paediatr Scand 1968; 57:401-4.
54. Perrone C E, Mattocks D A, Hristopoulos G, Plummer J D, Krajcik R A, Orentreich N. J Lipid Res 2008; 49:12-23.
55. Tsuboyama-Kasaoka N, Shozawa C, Sano K, et al. Endocrinology 2006; 147:3276-84.
56. Lemieux S, Despres J P. Diabete Metab 1994; 20:375-93.
57. Kumagai S, Kishimoto H, Masatakasuwa, Zou B, Harukasasaki. Metab Syndr Relat Disord 2005; 3:85-94.
58. Tozer R G, Tai P, Falconer W, et al. Antioxid Redox Signal 2008; 10:395-402.
59. Pacheco L, Guilford T, Kwyer T, Kongshavn PAL. J Human Virology 2002; 5.
60. Malloy V L, Krajcik R A, Bailey S J, Hristopoulos G, Plummer J D, Orentreich N. Aging Cell 2006; 5:305-14.
61. Czech M P, Fain J N. J Biol Chem 1972; 247:6218-23.
62. Olefsky J M. Horm Metab Res 1979; 11:209-13.
63. Kaur M, Kabra M, Das G P, Suri M, Verma I C. Indian Pediatr 1995; 32:1067-75.
64. Pogribna M, Melnyk S, Pogribny I, Chango A, Yi P, James S J. Am. J Hum Genet. 2001; 69:88-95.
65. Roizen N J, Patterson D. Lancet 2003; 361:1281-9.
66. Buchwald et al. Bariatric Surgery, A Systematic Review and Meta-analysis. JAMA. 2004; 292:1724-1737.
67. Maggard M A et al. Meta-analysis: surgical treatment of obesity. Ann Intern Med. 2005 Apr. 5; 142(7):547-59.
68. Fenton et al; The Molecular and Metabolic Bases of Inherited Disease. 7th ed. New York McGraw-Hill, 1995: 3129-49
69. Rosenblatt D. S The Molecular and Metabolic Bases of Inherited Disease. 7th ed. New York McGraw-Hill, 1995: 3111-28
70. Lejeune et al Ann Genet. 1992; 35:8-13

1.-45. (canceled)

46. A method of decreasing, or controlling, the amount of body fat mass in a subject at risk of, or requiring treatment for, being overweight or obese comprising administering a composition comprising an agent to said subject to inhibit or reduce the level of cysteine.

47. The method of claim 46, wherein the subject is obese and/or suffering from complications associated with obesity.

48. The method of claim 46, wherein the subject has a Body Mass Index (BMI) of over 25, and preferably over 30.

49. The method of claim 46 which comprises administering the agent orally, transdermally, intravenously or intradermally.

50. The method of claim 46, wherein the composition reduces the level of cysteine in the subject.

51. The method of claim 46, wherein the composition reduces the plasma concentration of cysteine in the subject.

52. The method of claim 46, wherein the composition reduces cysteine absorption.

53. The agent of claim 52, wherein the composition inhibits or reduces cysteine action on adipose tissue.

54. The method of claim 47, wherein the complication is selected from one or more of the following: cardiovascular disease, diabetes mellitus, dyslipidemias, metabolic Syndrome, musculoskeletal pains, arthritis, hypertension, pulmonary hypertension, atherosclerotic disease, congestive heart failure, cancer, breast cancer, uterine cancer, prostate cancer, sleep apnea syndrome, obesity hypoventilation syndrome, lower extremity edema, ventral/umbilical hernia, nonalcoholic steato-hepatitis, cholelithiasis, gastroesophageal reflux disease, stress urinary incontinence, psychosocial impairment or depression and polycystic ovarian syndrome.

55. The method of claim 46, wherein the composition reduces or inhibits the activity of cystathionine beta-synthase enzyme.

56. The method of claim 55, wherein the composition reduces or inhibits expression of a cystathionine beta-synthase gene.

57. The method of claim 46, wherein the composition reduces or inhibits the activity of cystathionine γ-lyase enzyme.

58. The method of claim 46, wherein the composition reduces or inhibits expression of a cystathionine γ-lyase gene.

59. The method of claim 46 further comprising reducing nutritional uptake by the subject.

60. The method of claim 46, wherein the composition reduces the subject's body fat mass by at least 2%.

61. The method of claim 46, wherein the agent is a nutraceutical.

62. The method of claim 60, wherein the composition reduces the subject's body fat mass by at least 5%.

63. The method of claim 62, wherein the composition reduces the subject's body fat mass by at least 10%.

64. The method of claim 46 wherein said agent is selected from a small molecule, an aptamer, a peptide, a nucleic acid, a polypeptide, an antibody and an antibody fragment.

65. The method of claim 64 wherein said agent is mesna.

66. The method of claim 65 wherein the composition further comprises ifosamide, or ifosamide and a dipeptidase inhibitor.

67. The method of claim 66 wherein said dipeptidase inhibitor is cilastatin.

68. A method of decreasing, or controlling, the amount of body fat mass in a subject at risk of, or requiring treatment for, being overweight or obese comprising administering a composition comprising mesna to said subject to inhibit or reduce the level of cysteine.

69. A method of predicting an increase in a subject's body fat mass comprising measuring the subject's plasma cysteine concentration, wherein a high cysteine concentration is associated with an increased risk of an increase in the subject's body fat mass.

70. A method of screening for an agent for the treatment of obesity comprising:
a. administering a test agent to an animal; and
b. detecting the plasma concentration of one or more sulphur containing amino acids.
